SEQUENCE LISTING (38 sequences)

SEQ ID NO: 1 - DNA, 22 bp, synthetic construct
  source 1..22: mol_type = other DNA
cggcacagtcattgaaagccta

SEQ ID NO: 2 - DNA, 21 bp, synthetic construct
  source 1..21: mol_type = other DNA
gttgctgatggcctgattgtc

SEQ ID NO: 3 - DNA, 24 bp, synthetic construct
  source 1..24: mol_type = other DNA
acggctgagtttcagtgagacctt

SEQ ID NO: 4 - DNA, 24 bp, synthetic construct
  source 1..24: mol_type = other DNA
aggtgtaaggtgctgatctgggtt

SEQ ID NO: 5 - DNA, 21 bp, synthetic construct
  source 1..21: mol_type = other DNA
gcttgaaggtgttgccctcag

SEQ ID NO: 6 - DNA, 20 bp, synthetic construct
  source 1..20: mol_type = other DNA
aagcctcgcgaccattcttg

SEQ ID NO: 7 - DNA, 20 bp, synthetic construct
  source 1..20: mol_type = other DNA
aggaaggtcacagccatagc

SEQ ID NO: 8 - DNA, 20 bp, synthetic construct
  source 1..20: mol_type = other DNA
cgatctctgccattttgacg

SEQ ID NO: 9 - DNA, 24 bp, synthetic construct
  source 1..24: mol_type = other DNA
agatggatgtgccaaacgtcctca

SEQ ID NO: 10 - DNA, 24 bp, synthetic construct
  source 1..24: mol_type = other DNA
aatatgcgaagcaccttggaagcc

SEQ ID NO: 11 - DNA, 22 bp, synthetic construct
  source 1..22: mol_type = other DNA
tggcagcagatggaaaacctag

SEQ ID NO: 12 - DNA, 21 bp, synthetic construct
  source 1..21: mol_type = other DNA
aggtacgaaatggccaggaca

SEQ ID NO: 13 - DNA, 20 bp, synthetic construct
  source 1..20: mol_type = other DNA
agatctcaccacagcccttc

SEQ ID NO: 14 - DNA, 20 bp, synthetic construct
  source 1..20: mol_type = other DNA
aacccttggtagggtgttca

SEQ ID NO: 15 - DNA, 28 bp, synthetic construct
  source 1..28: mol_type = other DNA
cgacttcaacagcaactcccactcttcc

SEQ ID NO: 16 - DNA, 27 bp, synthetic construct
  source 1..27: mol_type = other DNA
tgggtggtccagggtttcttactcctt

SEQ ID NO: 17 - DNA, 25 bp, synthetic construct
  source 1..25: mol_type = other DNA
ctcacttctgttcaggtgttatcca

SEQ ID NO: 18 - DNA, 21 bp, synthetic construct
  source 1..21: mol_type = other DNA
tccttttgccagtagatgcga

SEQ ID NO: 19 - DNA, 19 bp, synthetic construct
  source 1..19: mol_type = other DNA
tcctgagctgcgcctacag

SEQ ID NO: 20 - DNA, 20 bp, synthetic construct
  source 1..20: mol_type = other DNA
gcagggcaagtccacatctt

SEQ ID NO: 21 - DNA, 24 bp, synthetic construct
  source 1..24: mol_type = other DNA
ctgtaactccagctctgctccgta

SEQ ID NO: 22 - DNA, 24 bp, synthetic construct
  source 1..24: mol_type = other DNA
gcccataagtgtgctctgaagtga

SEQ ID NO: 23 - DNA, 20 bp, synthetic construct
  source 1..20: mol_type = other DNA
ccaacaccccacagaagtgc

SEQ ID NO: 24 - DNA, 20 bp, synthetic construct
  source 1..20: mol_type = other DNA
gccagcacctgctctgagac

SEQ ID NO: 25 - DNA, 21 bp, synthetic construct
  source 1..21: mol_type = other DNA
gaactgtacgctgtacctgca

SEQ ID NO: 26 - DNA, 21 bp, synthetic construct
  source 1..21: mol_type = other DNA
ttgatggccttcgattctgga

SEQ ID NO: 27 - DNA, 20 bp, synthetic construct
  source 1..20: mol_type = other DNA
tcagcctgagctacagatgc

SEQ ID NO: 28 - DNA, 20 bp, synthetic construct
  source 1..20: mol_type = other DNA
ctttagcttcgggtcaatgc

SEQ ID NO: 29 - DNA, 21 bp, synthetic construct
  source 1..21: mol_type = other DNA
tcagacttcagaaggacacgg

SEQ ID NO: 30 - DNA, 21 bp, synthetic construct
  source 1..21: mol_type = other DNA
ctgcatgattctgagcaggtg

SEQ ID NO: 31 - DNA, 21 bp, synthetic construct
  source 1..21: mol_type = other DNA
aagtgtgaatgtaaggtcccc

SEQ ID NO: 32 - DNA, 21 bp, synthetic construct
  source 1..21: mol_type = other DNA
gtgctccccttgttcagtatc

SEQ ID NO: 33 - DNA, 19 bp, synthetic construct
  source 1..19: mol_type = other DNA
aggtgactggggcattgat

SEQ ID NO: 34 - DNA, 20 bp, synthetic construct
  source 1..20: mol_type = other DNA
gcctccagcatgaaagtctc

SEQ ID NO: 35 - DNA, 21 bp, synthetic construct
  source 1..21: mol_type = other DNA
gctgcttgagccaactccata

SEQ ID NO: 36 - DNA, 19 bp, synthetic construct
  source 1..19: mol_type = other DNA
gaacgcggtacgacacctc

SEQ ID NO: 37 - DNA, 19 bp, synthetic construct
  source 1..19: mol_type = other DNA
acatcgctcagacaccatg

SEQ ID NO: 38 - DNA, 22 bp, synthetic construct
  source 1..22: mol_type = other DNA
tgtagttgaggtcaatgaaggg

(19) United States
(12) Patent Application Publication
(10) Pub. No.: US 2023/0405070 A1
(43) Pub. Date: Dec. 21, 2023

(54) PLECTRANTHUS AMBOINICUS EXTRACT FOR USE IN INHIBITING IMMUNE RESPONSES

(71) Applicant: Oneness Biotech Co., Ltd., Taipei City (TW)

(72) Inventors: Kung-Ming LU, Taipei City (TW); Min-Liang KUO, Taipei City (TW); Ching-Wen LIN, Taipei City (TW)

(21) Appl. No.: 18/324,745

(22) Filed: May 26, 2023

Related U.S. Application Data
(60) Provisional application No. 63/346,588, filed on May 27, 2022.

(51) Int. Cl. A61K 36/53; A61K 36/23; A61P 37/02

(52) U.S. Cl. CPC A61K 36/53; A61K 36/23; A61P 37/02

(57) ABSTRACT

A method for inhibiting immune responses, comprising administering to a subject in need thereof an effective amount of a pharmaceutical composition comprising salvigenin, and optionally cirsimaritin, rosmarinic acid, carvacrol, or a combination thereof.

### CROSS-REFERENCE TO RELATED APPLICATIONS

This application claims the benefit of priority to U.S. Provisional Patent Application No. 63/346,588, filed May 27, 2022, the disclosures of which are incorporated herein by reference in the entirety.

### REFERENCE TO SEQUENCE LISTING

The instant application contains a Sequence Listing which has been filed electronically in XML format and is hereby incorporated by reference in its entirety. Said XML copy, created on May 2, 2023, is named 112127-0167-0703US00_SEQ.XML and is 34,354 bytes in size.

### BACKGROUND OF THE INVENTION

Plectranthus amboinicus (also known previously or alternatively as Coleus amboinicus Lour., Coleus aromaticus Benth., Coleus aromaticus auct., Plectranthus aromaticus Roxb., Plectranthus aromaticus Benth., and Plectranthus amboinicus (Lour.) Spreng.), is a perennial medicinal herb of the Lamiaceae (also known as Labiatae) family native to Southern and Eastern Africa. Plectranthus amboinicus is also known as patchouli, Cuban oregano, Indian borage, Indian mint, Mexican mint, Mexican oregano, country borage, and Spanish thyme.

Centella asiatica (also known previously or alternatively as Centella asiatica Urban, Centella asiatica (L.) Urban, Hydrocotyle asiatica L., and Trisanthus cochinchinensis Lour.) is a perennial medicinal plant of the family Mackinlayaceae or subfamily Mackinlayoideae of the Apiaceae (also known as Umbelliferae) family native to Asia, Africa, and South America.

Centella asiatica is also known as European water-marvel, gotu kola, Kola, pennywort, Indian pennywort, marsh pennywort, pennyweed, Indian ginseng, Horse-hoof grass, Pegaga, Mandookaparni, Tiger herbal, Spadeleaf, or Tono. Extracts of Centella asiatica generally comprise two major compounds: asiaticoside and madecassic acid.

### SUMMARY OF THE INVENTION

The present disclosure is based, at least in part, on the unexpected discoveries that compositions comprising salvigenin, and optionally cirsimaritin, rosmarinic acid, carvacrol, or a combination thereof (e.g., Plectranthus amboinicus (PA) extract, optionally in combination with Centella asiatica (CA) extract) exhibited immune suppressive activities, e.g., suppressing the expression of CD86 and CD80, and altering the population of macrophage subtypes. It is further reported that the PA extract-containing composition disclosed herein alleviates psoriatic symptoms including erythema and plaque, suppressing immune activation pathways (e.g., the IL-17 and IL-23-mediated pathways), up-regulating the expression of skin barrier-related genes (e.g., filaggrin, loricrin, homerin, and desmocollin) and keratinocyte differentiation markers (e.g., K1), up-regulating pathways involved in attenuating autoimmune responses (e.g., the AhR signaling), and/or suppressing dendritic cell activation and maturation. Accordingly, the composition disclosed herein are expected to inhibit immune responses, thereby benefiting treatment of immune diseases such as autoimmune diseases, including, e.g., psoriasis, atopic dermatitis (e.g., intrinsic atopic dermatitis or extrinsic atopic dermatitis), asthma, allergic rhinitis, food allergy, hay fever, inflammatory bowel disease (IBD), and allergic contact dermatitis.

Accordingly, in some aspects, provided herein is a method for inhibiting immune responses, comprising administering to a subject in need thereof an effective amount of a composition (e.g., a pharmaceutical composition) comprising salvigenin and a pharmaceutically acceptable carrier. In some embodiments, the pharmaceutical composition further comprises cirsimaritin, rosmarinic acid, carvacrol, or a combination thereof.

In some embodiments, the composition comprises a Plectranthus amboinicus (PA) extract. In some instances, the PA extract is prepared by a process comprising: (i) mixing a part of PA (e.g., an above-ground part) with an extracting solution to produce a first PA extract, (ii) filtrating and concentrating the first PA extract to produce a concentrated PA extract; (iii) contacting the concentrated PA extract onto a hydrophobic interaction chromatography resin, and (iv) eluting the column with a eluent solution to product the PA extract. In some examples, the extracting solution comprises a solvent having a polarity index of about 2.9 to 6.6; optionally wherein the extracting solution is acetone, butyl methyl ether, ethanol, ethyl acetate, isopropyl alcohol, methanol, or a mixture thereof. In some examples, the eluent solution comprises a solvent having a polarity index of about 2.1-5.4; optionally wherein the eluent solution comprises a mixture of at least two solvents selected from the group consisting of acetone, ethanol, ethyl acetate, and hexane.

In some embodiments, the composition further comprises asiaticoside. For example, the composition further comprises a Centella asiatica (CA) extract, which comprises the asiaticoside.

In some embodiments, the subject to be treated by any of the methods disclosed herein may be a human patient having or suspected of having an autoimmune disease. In some examples, the autoimmune disease is psoriasis. In other examples, the autoimmune disease can be inflammatory bowel disease (IBD), e.g., Crohn's disease or ulcerative colitis. Alternatively, the subject to be treated by the methods disclosed herein may be a human patient having or suspected of having an immune disorder, e.g., an allergic disorder. Examples include, but are not limited to, atopic dermatitis (e.g., intrinsic atopic dermatitis or extrinsic atopic dermatitis), asthma; allergic rhinitis; food allergy; or hay fever.

Also within the scope of the present disclosures are the composition as disclosed herein for use in inhibiting immune responses (e.g., treating an autoimmune disease) in a subject in need thereof, and the composition for use in manufacturing a medicament for the intended medical uses.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic depicting potential molecular pathways for ON101 action during diabetic wound healing. Topical ON101 treatment directly reduces the proportion of M1 macrophages (CD86⁺ or CD80⁺) and enriches the M2 populations by inhibiting proinflammatory cytokines. Under such a circumstance, ON101 can further stimulate GCSF and CXCL3 production/secretion from surrounding cells, including macrophages and ADPCs, adding additional force to drive the polarization of M2a and M2c macrophages. Thus, topical ON101 application alters the macrophage subtype from an M1- to an M2-dominant distribution in diabetic mice leading to an accelerated wound healing.

FIGS. 2A-2E show IHC staining and quantification of serial sections of biopsies from ON101⁻ or placebo-treated wounds. Depicted are images of IHC results for anti-Ly-6C/6G (FIG. 2A), anti-CD3 (FIG. 2B), anti-MOMA2 (FIG. 2C), anti-MMP9 (FIG. 2D), and anti-keratin 14 (K14; FIG. 2E) within wound biopsies of db/db mice at the indicated time points. Quantified results for each antibody are shown at right (except for anti-K14) and are expressed as means±SEMs (n=8 in per group). Scale bar=250 μm.

FIGS. 3A-3C show that ON101 alters the population of iNOS⁺, CD163⁺, and CD206⁺ cells in diabetic wound surroundings by immunohistochemical staining of wound biopsies from db/db mice. FIG. 3A: quantification of iNOS-positive stain in the whole field compared with the total tissue area; n=8 selected fields per group. FIG. 3B: quantification of the CD163-positive stain in the dermis compared with total nuclear stain, n=16 selected fields per group. FIG. 3C: quantification of the CD206-positive stain in the wound edge compared with the nuclear stain in the field. n=8 selected fields per group. All values represent means±SEM. Statistical analysis is based on the Student's t-test; *P<0.05, **P<0.01, ***P<0.001.

FIGS. 4A-4E show that ON101 alters the dynamics of macrophage subtypes and the transcriptional expression profile of inflammation-associated cytokines in db/db mice. FIG. 4A shows the number of F4/80-positive macrophages around wound beds in db/db mice after treatment with ON101 or placebo cream by FACS analysis. A fixed number of cells (5×10⁴) was gated, and the F4/80-positive population was quantified after excluding dead cells using 7-AAD viability staining. FIG. 4B shows the quantification of the percentage of CD163⁺ cells among F4/80⁺ cells in each wound by FACS analysis. FIG. 4C shows the ratio of cell numbers in the non-M2 population/M2⁺ population in each wound sample by FACS analysis. FIGS. 4D-4E show relative expressions (normalized with GAPDH and compared to day 0) of pro-inflammatory cytokine genes (FIG. 4D) and macrophage-associated cytokine genes (FIG. 4E) in ON101⁻ or placebo cream-treated wounds in db/db mice, determined by qRT-PCR (n=4 mice per group). Gene expression levels were normalized to those of endogenous GAPDH. Data are shown as means±SEM. Data are shown as means±SEM; Student's t-test, *P<0.05, **P<0.01, and ***P<0.001.

FIGS. 5A-5I show that ON101 directly attenuates M1 markers under high-glucose condition. FIGS. 5A-5D show THP-1-derived M1 (FIGS. 5A-5B) and M2 (FIGS. 5C-5D) polarization models in culture medium containing normal glucose (NG; 1,000 mg/L glucose) or high glucose (HG; 4,500 mg/L glucose), as determined by flow cytometry. FIG. 5A shows representative histograms showing CD86 (upper panel) and CD80 (lower panel) intensity cultured under M1-polarizing conditions in NG or HG medium. FIG. 5B is a summary of data calculated from three independent experiments showing mean fluorescence intensities (MFIs) for CD86 and CD80. FIG. 5C is representative histograms showing CD163 (upper panel) and CD206 (lower panel) expression intensities under M2-polarizing conditions in NG or HG medium. FIG. 5D is a summary of data calculated from three independent experiments showing MFIs for CD163 and CD206. Data are shown as means±SEM. Statistical analysis is based on the Student's t-test. *P<0.05, **P<0.01, ***P<0.001). MFI values (FIG. 5E) and fold changes in the gene expressions (FIG. 5F) of CD86 and CD80 induced by ON101 in M1 macrophages (compared with 0 ug/ml of ON101). FIG. 5G shows cell viability assays of THP-1-derived M0, M1, and M2 macrophages after ON101 treatment for 48 h. The results were standardized against the control group for each cell type. FIG. 5H is MFIs of CD163 (left panel) and CD206 (right panel) after a 48-h treatment with an M2 polarizing cocktail and different concentrations of ON101 as indicated. Data are shown as means±SEMs of three independent experiments. FIG. 5I is M1 macrophages derived from human PBMCs treated with ON101 for 96 h. Left panel: CD86; right panel: CD80. Markers were analyzed by FACS (n=6; paired Student's t test). Data are shown as means±SEM; Student's t-test, **P<0.01, ***P<0.001.

FIGS. 6A-6C show ON101 modulate M1 associated cytokine release and gene expressions under high-glucose condition. FIG. 6A is an experimental design and data analysis flow chart to reveal gene expression profiles in M1 macrophages following ON101 treatment evaluated by RNA sequencing. FIG. 6B shows cytokine/chemokine gene expressions altered by 48 h of ON101 treatments in M1 macrophages analyzed by q-RT-PCR. DMSO serves as a control treatment. Data are shown as means±SEMs of three independent experiments. FIG. 6C shows relative levels of secreted pro-inflammatory cytokines measured by ELISA at 24 h for IL-6 (left panel) and TNF-α (middle panel) and 96 h for IL-1β (right panel) after M1-polarizing (n=6 individual donors; paired Student's t test). Data are shown as means±SEM; **P<0.01, ***P<0.001.

FIGS. 7A-7C show that ON101 attenuates M1-mediated M2a marker expression. FIG. 7A shows a schematic depicting the experimental flowchart. CM: conditioned medium. MFI values for CD163⁺ (right panel) and CD206⁺ (right panel) M2 macrophage populations (FIG. 7B) and CD80⁺ (right panel) and CD86⁺ (left panel) M1 macrophage markers (FIG. 7C) under M2 polarization condition were analyzed by flow cytometry 48 h after M2 polarization. Data are shown as means±SEM of three experiments. Statistical analysis is based on the Student's t-test. **P<0.01; ***P<0.001.

FIGS. 8A-8I show that ON101 promotes M1-to-M2 macrophage transition through ADPC-mediated production/secretion of GCSF and CXCL3. FIG. 8A show immunohistochemical detection of Pref-1 in wound biopsies from db/db mice and quantified results using an area quantification module with 16 selected fields per group. Scale bar=100 μm. FIG. 8B shows a schematic of CM collected from ADPCs treated with ON101 for 24 h for a second round of M1 polarization. MFIs for M1 markers (FIG. 8C; left panel: CD80; right panel: CD86) and M2 markers (FIG. 8D; left panel: CD163; right panel: CD206), detected by FACS 48 h after M1 polarization. Expression of GCSF and CXCL3 genes (FIG. 8E) or other indicated cytokines (FIG. 8F) in human ADPCs after 24 h of ON101 treatment, determined by qRT-PCR. Expression of CD163 and CD206 following co-administration of recombinant GCSF (FIG. 8G) or CXCL3 (FIG. 8H) protein and corresponding antibodies with M1-polarizing cytokines for 48 h. Data shown as means±SEM of three experiments. FIG. 8I shows immunoblots of p-Stat3, total Stat3, and p-Akt, total Akt upon treatments. β-actin was used as a protein loading control. Ab, antibody; Anti-GCSF: 1 ug/ml; anti-CXCL3: 50 ng/ml. All statistical data was conducted by Student's t-test, *P<0.05;**P<0.01; ***P<0.001.

FIGS. 9A-9H show that M2 macrophages are required for normal as well as ON101-enhanced diabetic wound healing. FIGS. 9A-9C show subcutaneous injection of m-Clo (m-Clodrosome) or m-Enc (m-Encapasome) during normal wound healing in C57BL/6 mice. Schematic in (FIG. 9A); n=4 mice per group. (FIG. 9B) Wound recovery rate, calculated as the percentage change from the original size (day 0) and images of wounds at the indicated times. Upper panel: a chart showing would recovery rates. Lower panel: photos showing wound recovery images. Biopsies from the point of wounding were analyzed by flow cytometry to detect the proportions of M2 macrophages (F4/80⁺/CD163⁺) at indicated time point. (FIG. 9C) Data represent the percentages of F4/80⁺ or CD163⁺ macrophages. FIGS. 9D-9H show M2 macrophage dependence of ON101-promoted diabetic wound healing. m-Clo/m-Enc. Schematic in (FIG. 9D); n=4 mice per group. (FIG. 9E) Wound recovery rate (percentage change from day 0) and images of wounds at the indicated times for each treatment group. Upper panel: a chart showing would recovery rates. Lower panel: photos showing wound recovery images. (FIG. 9F) H&E staining represent re-epithelization (dotted line) and the wound bed (WB) in the indicated treatment. (FIG. 9G) FACS analysis of the proportions of M2 macrophages (F4/80⁺/CD163⁺) in wound surroundings. (FIG. 9H) IHC staining of wound biopsies with indicated antibodies on day 9 after wounding. #, wound bed. Scale bar=250 μm. Data represent means±SEM; P values in (FIG. 9B) and (FIG. 9E) were analyzed using two-way repeated measures ANOVA whereas FIG. 9G was analyzed using Student's t-test (*P<0.05, **P<0.01, ***P<0.001). NS, not significant.

FIGS. 10A-10C include diagrams showing that a Plectranthus amboinicus (PA) extract PA-F4 alleviates Imiquimod (IMQ)-induced psoriatic symptoms in a mouse model. FIG. 10A: a diagram illustrating an exemplary study design. FIG. 10B: photos showing effects of the 0.5% PA-F4 cream and 1% PA-F4 cream on IMQ-induced psoriasis on mouse skin. FIG. 10C: charts showing the effects of the PA-F4 cream on IMQ-induced psoriatic symptoms, including erythema (left panel), plaque (middle panel), and both erythema and plaque (right panel).

FIGS. 11A-11D include diagrams showing the dose-dependent suppressive effects of the PA-F4 cream on the expression levels IL-17a (FIG. 11A), IL-17f (FIG. 11B), mIL-23 (FIG. 11C), and IL-22 (FIG. 11D) in IMQ-treated skin tissues.

FIGS. 12A-12E includes diagrams showing that PA-F4 up-relates the expression levels of filaggrin (FIG. 12A), loricrin (FIG. 12B), homerin (FIG. 12C), desmocollin (FIG. 12D), and Involucrin (FIG. 12E) in IMQ-treated skin tissues in a dose-dependent manner.

FIG. 13 is a chart showing that PA-F4 up-relates the expression level of keratin 1 (K1) in IMQ-treated skin tissues in a dose-dependent manner.

FIG. 14 is a chart showing that PA-F4 and ON101 compositions up-regulate aryl hydrocarbon receptor (AhR) activity in a dose dependent manner in CaCo2 cells stably expressing an AhR-response element.

FIG. 15 is a chart showing that PA-F4 suppresses dendritic cell (DC) activation and maturation as demonstrated by the reduction of the CD11c⁺ to CD86⁺ ratios.

FIGS. 16A-16D include diagrams showing PA-F4 upregulates inolucrin as observed in an in vitro primary keratinocyte stimulation experiment. FIG. 16A: Involucrin. FIG. 16B: Filaggrin. FIG. 16C: HRNR. FIG. 16D: Loricrin.

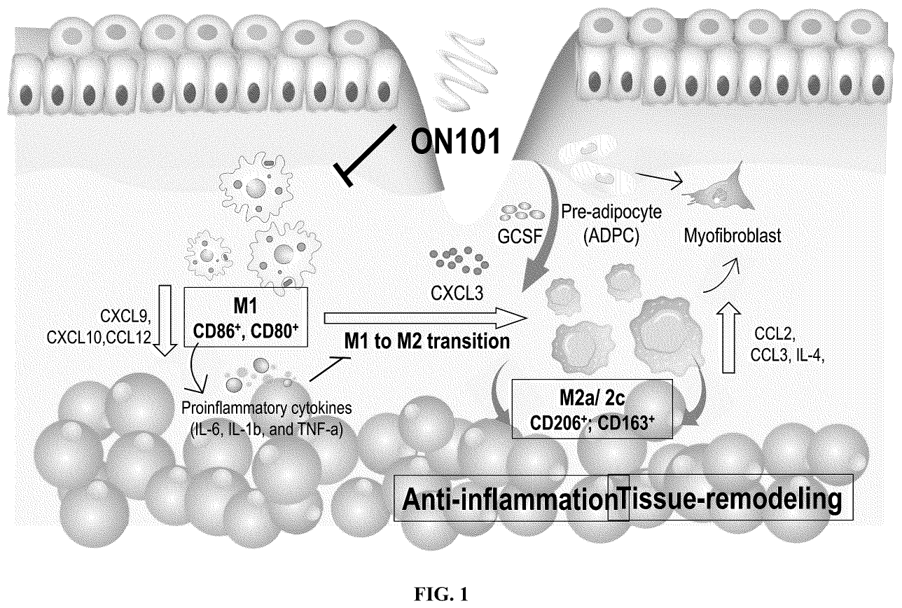

### DETAILED DESCRIPTION OF THE INVENTION

Provided herein are pharmaceutical compositions comprising Plectranthus amboinicus extract or active agents thereof, and optionally Centella asiatica extract or active agents thereof, for use in modulating immune responses (e.g., suppressing immune cell activation such as T cell activation). It is reported herein that exemplary compositions showed unexpected activity in modulating the population of macrophage subtypes and suppression expression of immune cell receptors such as CD80 and CD86, which are ligands of co-stimulatory molecule CD28. Decreasing the expression level of CD80 and/or CD86 would be expected to down-regulate co-stimulation of T cells via the CD28-mediated co-stimulatory signaling, and thus down-regulate immune responses. Additionally, it is reported that the exemplary compositions showed unexpected activity in modulating the development of autoimmune responses and diseases, such as alleviating psoriatic symptoms including erythema and plaque, suppressing immune activation pathways (e.g., IL-17 and IL-23), up-regulating the expression of skin barrier-related genes (e.g., filaggrin, loricrin, homerin, and desmocollin) and keratinocyte differentiation markers (e.g., K1), up-regulating pathways involved in attenuating autoimmune responses (e.g., AhR signaling), and suppressing dendritic cell activation and maturation.

Accordingly, the pharmaceutical compositions disclosed herein would be expected to suppress immune responses, via, e.g., suppressing the expression of CD80 and/or CD86, enhancing the population of anti-inflammatory CD163⁺ or CD206⁺ macrophages/monocytes, reducing the expression of CXCL9, CXCL10, and CXCL11; alleviating psoriatic symptoms, suppressing IL-17 and IL-23-mediated immune activation, up-regulating the expression of filaggrin, loricrin, homerin, desmocollin, and/or K1, up-regulating the AhR signaling pathway, and/or suppressing dendritic cell activation and maturation. Such pharmaceutical compositions would be expected to benefit treatment of immune diseases such as autoimmune diseases, (e.g., psoriasis, and inflammatory bowel disease (IBD) such as ulcerative colitis or Crohn's disease), atopic dermatitis (e.g., intrinsic atopic dermatitis or extrinsic atopic dermatitis), asthma, allergic rhinitis, food allergy, hay fever, and allergic contact dermatitis.

### I. Compositions

The instant disclosure herein provides a composition such as a pharmaceutical composition for modulating (e.g., inhibiting) immune responses and/or for treating immune disorders such as autoimmune diseases. The compositions disclosed herein may comprise a PA extract or one or more active agents contained therein (e.g., salvigenin, and optionally cirsimaritin, rosmarinic acid, carvacrol, or a combination thereof). In some embodiments the pharmaceutical composition may further comprise a CA extract or one or more active agents contained therein (e.g., asiaticoside).

(i) Active Agents

The active agent in the composition described herein comprises salvigenin. In some embodiments, the active agent may further comprise cirsimaritin, rosmarinic acid, carvacrol, or a combination thereof. In some instances, the composition comprises a PA extract, which comprises salvigenin and optionally one or more of cirsimaritin, rosmarinic acid, and carvacrol.

Plectranthus amboinicus Extract

A PA extract refers to an extract obtained from PA plant using one or more suitable solvents. In some examples, the PA extract is prepared using an above-ground part of the PA plant. In some instances, at least one of the solvents used for preparing the extract has a polarity index lower than 7 (e.g., less than 5). See, e.g., U.S. Pat. No. 10,758,584, the relevant disclosures of which are incorporated by reference for the subject matter and purpose referenced herein. As used herein, a solvent refers to a substance or a mixture of substances that dissolves another to form a solution. A PA extract as described herein may be prepared using one solvent. The solvent used in each extracting step for preparing the extracts described herein (including both PA and CA extracts) may be a single solvent. Alternatively, it can be a mixture of two or more solvents.

The PA extract described herein may comprise terpenoids (e.g., monoterpenoids, diterpenoids, triterpenoids, and/or sesquiterpenoids), flavonoids, phenolics, essential oils, or a combination thereof. The PA extract for making the pharmaceutical compositions disclosed herein may comprise salvigenin, and optionally one or more of cirsimaritin, rosmarinic acid, and carvacrol.

The PA extract described herein may be prepared by extracting a whole PA plant or a part thereof (e.g., an above-ground part) with one or more suitable solvents to produce a solution and then drying the solution to produce the PA extract. Since the PA extract comprises flavonoids, terpenoids (e.g., monoterpenoids, diterpenoids, triterpenoids, and/or sesquiterpenoids), phenolics, or essential oils, which are non-polar molecules, at least one of the extracting solvents may have a relatively low polarity (e.g., having a polarity index lower than 7) to facilitate dissolution of the non-polar molecules. “Extracting” can be performed by either contacting the PA material directly with a suitable solvent or by eluting active components of PA from resins on which the active components are attached.

In some examples, a solvent having a polarity index lower than 7 can be used for extracting the active components from PA to produce the PA extract. Such a solvent can be ethyl acetate, methyl acetate, propanol, butanol, or chloroform. Alternatively, the solvent can be a mixture of one or more solvents having different polarity index. Examples include, but are not limited to, a mixture of ethanol and ethyl acetate, ethyl acetate and butanol, ethanol and propanol, methyl acetate and butanol.

In some embodiments, the PA extract may be prepared by a process involving the use of one solvent, such as a solvent having a polarity index lower than 7 (e.g., <about 6.5, <about 6.0; <about 5.5, <about 5.0, <4.9, <4.8, <4.7, <4.6, or <4.5). Examples include, but are not limited to, methanol, ethanol, acetone, ethyl acetate, butanol, dichloromethane, or a combination thereof.

PA materials, which can be a whole PA or a part thereof (e.g., an above-ground part such as leaves), can be prepared by routine methodology. The PA material can be a fresh plant or a part thereof. Alternatively, the PA material can be in dried form. The PA can optionally be dried to form powders, which can be used as the PA material for preparing the PA extract.

Any of the PA materials as described herein can be extracted, one or more times, by a suitable solvent to produce a crude extract. The solvent for use in preparing the crude extract may be a high-polarity solvent, for example, having a polarity index above 5 and preferably below 7 (e.g., >5.2; >5.5, >5.8, >6 or above and preferably below 7). Examples include, but are not limited to, ethanol, acetone, methanol, water, or a combination thereof. If necessary, the crude extract can be concentrated by a conventional method to produce a concentrated crude extract.

The crude extract can then be brought in contact with a suitable resin (e.g., a non-ionic absorbent resin) under suitable conditions that allow for binding of active components in the crude extract onto the resin. Exemplary resins for use in preparing the Plectranthus amboinicus extract include, but are not limited to, DIAION® HP20, DIAION® HP20SS, Sepabeads® SP207, Amberlite™ XAD-2, or Amberlite™ XAD-4.

Afterwards, the resin can be washed one or more times and eluted with a suitable solvent, for example, a solvent having a polarity index lower than 7, to produce a PA extract, which can then be dried by a conventional method (e.g., freeze-drying, spray-drying, or concentration drying) to produce dried the PA extract, which can be in semisolid or paste form.

In some examples, the resin absorption step can be performed by mixing the crude extract with the resin in a container. In other embodiments, the resin separation step can be performed by a chromatography column setting.

In one example, an extract of PA can be prepared as follows. An overground part of PA (about 1.5 g), including leaves and/or stems, can be collected and extracted with a solvent having a polarity less than 7 (e.g., methanol, ethanol, acetone, ethyl acetate, butanol, dichloromethane, or a combination thereof) at room temperature for 30 min to 6 hours. Alternatively, this extraction process can be carried out at a temperature of about 50 to 80° C. The resultant crude extract can be directly loaded to a non-ionic adsorbent resin column and eluted by a solvent having a polarity less than 6 (e.g., ethanol, ethyl acetate, butanol, dichloromethane, hexane, toluene, or a combination thereof). The eluted components can be collected and purified by extraction with a solvent having a polarity less than 6 (e.g., those described herein). The resultant filtrate can be collected to produce a PA extract.

Centella asiatica Extract

In some embodiments, the composition such as the pharmaceutical composition disclosed herein may further comprise asiaticoside. In some instances, the composition further comprises a CA extract, which may comprise the asiaticoside compound. The CA extract as described herein refers to an extract obtained from whole CA plants or a part thereof. The CA extract may comprise asiaticoside, and optionally madecassic acid.

The CA extract can be prepared following a conventional method, for example, those described in U.S. Pat. Nos. 5,834,437, 6,417,349, 6,475,536, and 6,267,996, CN 1313124, CN 1089497, and CN 1194154. Below is an example.

CA materials can be prepared via routine practice. Such materials can be fresh CA plant or a part thereof, or dried CA. The CA materials can be extracted with a suitable solvent such as water, ethanol, or a mixture thereof, to produce a crude extract. The crude extract, which can be optionally concentrated, can be either mixed with a suitable resin or loaded onto a column packed with the resin. After being washed for one or more times, the resin can be eluted with a suitable solvent. The resultant eluent can be concentrated to form a paste, which can be dried by a conventional method, for example, vacuum dry, to produce powders of the CA extract. When necessary, the CA powder can be grinded through a mesh (e.g., a No. 100 mesh).

An extract of CA can be prepared by the same or similar process as described above for making the PA extract. The PA Extract and/or CA Extract may be concentrated using a pressure-reducing rotary evaporator.

(ii) Pharmaceutical Compositions

Any of the active compounds (e.g., salvigenin, and optionally one or more of cirsimaritin, rosmarinic acid, carvacrol, and/or asiaticoside), the PA extract, optionally in combination with the CA extract as disclosed herein can be mixed with a suitable carrier (e.g., a pharmaceutically acceptable carrier) to form a composition (e.g., a pharmaceutical composition) for use in modulating (e.g., inhibiting) immune responses and treating a target disease as disclosed herein. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The compositions such as pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™ PLURONICS™ or polyethylene glycol (PEG).

In some examples, the pharmaceutical composition described herein comprises liposomes containing the active agent, which can be prepared by methods known in the art or such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active agent may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the active agents, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The compositions such as the pharmaceutical composition to be used for in vivo administration must be sterile. This is readily accomplished by, for example, filtration through sterile filtration membranes. The compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing the active agents with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### II. Methods for Modulating Immune Responses

To practice the method disclosed herein, an effective amount of the composition (e.g., the pharmaceutical composition) described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having an immune disorder, such as an autoimmune disease. Examples of target immune disorders include, but are not limited to, psoriasis, atopic dermatitis (e.g., intrinsic or extrinsic), asthma, allergic rhinitis, food allergy, hay fever, contact dermatitis, and inflammatory bowel disease (IBD) (e.g., Crohn's disease or ulcerative colitis).

Psoriasis is a long-lasting, autoimmune disease characterized by raised areas of abnormal skin. It is a T cell-mediated autoimmune disorder, resulting from the interaction between multiple genetic and environmental factors. There is no known cure for psoriasis. Some treatments can help control or alleviate the symptoms, for example, steroid creams, vitamin D3 cream, ultraviolet light, and immunosuppressive drugs, such as methotrexate.

Atopic dermatitis (AD), also known as atopic eczema, is a long-term type of inflammation of the skin (dermatitis). It results in itchy, red, swollen, and cracked skin. The cause of AD is unknown but believed to involve genetics, immune system dysfunction, environmental exposures, and difficulties with the permeability of the skin. AD can be categorized into the extrinsic and intrinsic types. Extrinsic or allergic AD shows high total serum IgE levels and the presence of specific IgE for environmental and food allergens, whereas intrinsic or non-allergic AD exhibits normal total IgE values and the absence of specific IgE. There is currently no cure for AD. Treatment of AD typically involves avoiding things that make the condition worse, enhancing the skin barrier through skin care and treating the underlying skin inflammation.

Asthma is a long-term inflammatory disease of the airways of the lungs. It is characterized by variable and recurring symptoms, reversible airflow obstruction, and easily triggered bronchospasms. Symptoms include episodes of wheezing, coughing, chest tightness, and shortness of breath. Asthma is a result of an overactive immune response, believed to be caused by a combination of complex and incompletely understood environmental and genetic interactions. There is no known cure for asthma, but it can be controlled. Symptoms can be prevented by avoiding triggers, such as allergens and respiratory irritants, and suppressed with the use of inhaled corticosteroids.

Allergic rhinitis, of which the seasonal type is called hay fever, is a type of inflammation in the nose that occurs when the immune system overreacts to allergens in the air. Signs and symptoms include a runny or stuffy nose, sneezing, red, itchy, and watery eyes, and swelling around the eyes. Allergic rhinitis is typically triggered by environmental allergens such as pollen, pet hair, dust, or mold. Inherited genetics and environmental exposures contribute to the development of allergies The underlying mechanism involves IgE antibodies that attach to an allergen, and subsequently result in the release of inflammatory chemicals such as histamine from mast cells. It causes mucous membranes in the nose, eyes and throat to become inflamed and itchy as they work to eject the allergen. Treatment is mainly to prevent or reduce the symptoms caused by the inflammation of affected tissues. Nasal glucocorticoids (steroids) delivered by a nasal spray are the first-line treatment for the symptoms of allergic rhinitis.

Food allergy is an abnormal immune response to food (“food allergens”). The most common food allergens include milk, eggs, peanuts, tree nuts, fish, shellfish, soy, and wheat, which are often referred to as “the big eight’. The symptoms of food allergy may range from mild to severe, including itchiness, swelling of the tongue, vomiting, diarrhea, hives, trouble breathing, or low blood pressure. There is presently no cure for food allergy. Currently, healthcare providers can manage patients' food allergies by encouraging them to avoid foods that may cause an allergic reaction and by treating severe reactions when they arise.

Hay fever is the seasonal type of allergic rhinitis, see discussion above. Hay fever is a common term used to describe allergic rhinitis because it tends to strike at hay season, when farmers harvest hay, which typically lasts from late spring to late summer or early fall.

Contact dermatitis is an itchy rash caused by direct contact with a substance that triggers an allergic reaction. Many substances can cause this reaction, such as cosmetics, fragrances, jewelry and plants. The rash often shows up within days of exposure. Identifying and avoiding substances that cause the allergic reaction may reduce the risk of contact dermatitis.

Inflammatory bowel disease (IBD) refers to a group of disorders that cause inflammation of the digestive tract. Crohn's disease and ulcerative colitis are the two major types of IBD. Inflammatory bowel disease symptoms vary, depending on the severity of inflammation and where it occurs. T cells play a pivotal role in the immune response underlying IBD pathogenesis. Current treatment for IBD involves a combination of self-care and medical treatment to alleviate symptoms.

A subject having a target autoimmune disease can be identified by routine medical examinations. In some embodiments, the subject to be treated by the method described herein may be a human patient having an immune disorder, e.g., an autoimmune disease or an allergic disorder, such as those disclosed herein. Such a human patient may have undergone or is undergoing a therapy for the autoimmune disease.

A subject suspected of having any of such target disease/disorder might show one or more symptoms of the immune disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the composition achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the active agent. Frequency of administration may be determined and adjusted over the course of therapy, and is generally, but not necessarily, based on treatment and/or suppression and/or amelioration and/or delay of a target disease/disorder. Alternatively, sustained continuous release formulations of the composition may be appropriate. Various formulations and devices for achieving sustained release are known in the art.

In one example, dosages for a composition as described herein may be determined empirically in individuals who have been given one or more administration(s) of the composition. Individuals are given incremental dosages of the agonist. To assess efficacy of the agonist, an indicator of the disease/disorder can be followed.

Generally, for administration of any of the compositions described herein, an initial candidate dosage can be given to a subject. For repeated administrations over several days or longer, depending on the condition, the treatment is sustained until a desired suppression of symptoms occurs or until sufficient therapeutic levels are achieved to alleviate a target disease or disorder, or a symptom thereof. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of the composition as described herein will depend on the specific active agent contained therein, the type and severity of the disease/disorder, whether the composition is administered for preventive or therapeutic purposes, previous therapy, the patient's clinical history and response to treatment, and the discretion of the attending physician. Typically, the clinician will administer the composition, until a dosage is reached that achieves the desired result. In some embodiments, the desired result is an increase in a suppression of immune responses in the patient. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of the composition may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethyllactamide, dimethylformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the composition and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the active agent, can be dissolved and administered in a pharmaceutical excipient such as Water-for-Injection, 0.9% saline, or 5% glucose solution.

In one embodiment, a composition as disclosed herein is administered via site-specific or targeted local delivery techniques. Examples of site-specific or targeted local delivery techniques include various implantable depot sources of the composition or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

The particular dosage regimen, i.e., dose, timing and repetition, used in the method described herein will depend on the particular subject and that subject's medical history.

In some embodiments, any of the compositions disclosed herein may be co-used with one or more additional therapeutic agents for treating the target disease. In some instances, the additional therapeutic agents may serve to enhance and/or complement the effectiveness of the composition disclosed herein.

Treatment efficacy for a target disease/disorder can be assessed by methods well-known in the art.

### III. Kits For Use in Modulating Immune Responses

The present disclosure also provides kits for use in modulating immune responses and/or for treating a target disease such as those disclosed herein. Such kits may include one or more containers comprising a composition as described herein, which comprises a Plectranthus amboinicus extract or active agents thereof, and optionally a Centella asiatica extract or active agents thereof.

In some embodiments, the kit may comprise instructions for use in accordance with any of the methods described herein. The included instructions may comprise a description of administration of the composition to modulate immune responses to any of the methods described herein. The kit may further comprise a description of selecting an individual suitable for treatment based on identifying whether that individual has wounds in need of treatment.

The instructions relating to the use of the composition disclosed herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for modulating immune responses and/or treating a target disease. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. At least one active agent in the composition is salvigenin or a PA extract comprising such. Additional active agents such as those disclosed herein may be included, for example, a CA extract.

Kits may optionally provide additional components such as interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1. ON101 Restores Pro-Healing and Remodeling-Associated M2a/c Macrophages

Diabetic wounds exhibit chronic inflammation and delayed tissue proliferation or remodeling, mainly due to prolonged pro-inflammatory (M1) macrophage activity and defects in transition to pro-healing/pro-remodeling (M2a/M2c; CD206⁺ and/or CD163⁺) macrophages. Topical treatment with ON101, a plant-based potential therapeutic for diabetic foot ulcers, increased M2c-like (CD163⁺ and CD206⁺) cells and suppressed M1-like cells, altering the inflammatory gene profile in a diabetic mouse model compared to controls. An in vitro macrophage-polarizing model revealed that ON101 directly suppressed CD80⁺ and CD86⁺ M1-macrophage polarization and M1-associated pro-inflammatory cytokines at both protein and transcriptional levels. Notably, conditioned medium (CM) collected from ON101-treated M1 macrophages reversed the M1-CM-mediated suppression of CD206⁺ macrophages. Furthermore, CM from ON101-treated adipocyte progenitor cells (ADPCs) significantly promoted CD206⁺ and CD163⁺ macrophages but strongly inhibited M1-like cells. ON101 treatment also stimulated the expression of GCSF and CXCL3 genes in human ADPCs. Interestingly, treatment with recombinant GCSF protein enhanced both CD206⁺ and CD163⁺ M2 markers, whereas CXCL3 treatment only stimulated CD163⁺ M2 macrophages. Depletion of cutaneous M2 macrophages inhibited ON101-induced diabetic wound healing. Thus, ON101 directly suppressed M1 macrophages and facilitated the GCSF- and CXCL3-mediated transition from M1 to M2 macrophages, lowering inflammation and leading to faster diabetic wound healing.

### Materials and Methods

### Formulation

ON101, a topical cream formulated by using identified, defined fractions of Plectranthus amboinicus (PA-F4) and Centella asiatica (Si) in a proprietary ratio, has been reported to be capable of inhibiting NLRP3 inflammasome signaling and regulating macrophages (Huang et al., JAMA Netw Open, 2021; Leu et al., Front Pharmacol, 2019). Having established the clinical efficacy of ON101 in promoting wound healing in randomized controlled studies (Huang et al., JAMA Netw Open, 2021; Leu et al., Front Pharmacol, 2019), described herein is a further exploration of the molecular mechanism by which ON101 regulates cell function and cellular networks to improve healing in diabetic wounds and gain insight into the macrophage phenotypes involved in DFUs.

ON101 cream and placebo cream were used in animal experiments. ON101 cream contains 1.25% of ON101 powder and was manufactured as previously described (Huang et al., JAMA Netw Open, 2021). Placebo cream was the cream-based vehicle control containing no ON101. For in vitro assays, ON101 powder was dissolved in DMSO (D12345, Thermo Fisher) at a stock concentration of 25 mg/ml.

### Animal Models of Diabetes

All animal experiments were approved by the Institutional Animal Care and Use Committee (IACUC) of Oneness Biotech Co., Ltd., Taiwan. Mice were housed in standard conditions of 25° C. with a 12/12-h light-dark cycle with ad libitum access to food and water. Male leptin receptor-deficient (db/db) mice aged 9 wks (C57BLKS/J Iar−+Lepr^{db}/+Lepr^{db}, Institute for Animal Reproduction, Ibaraki, Japan) with fasting blood glucose levels of 300-500 mg/dL and body weight >35 g were assigned randomly to groups for drug treatments. Two full-thickness wounds were created on the back of each mouse using a sterile 6-mm biopsy punch. The wounds were covered with silicone splints (inner/outer diameter, 10/14 mm) to anchor them and reduce skin contraction, and then covered with a transparent occlusive dressing (Tegaderm; 3M). ON101 (1.25%) or a placebo cream were applied topically once daily from day 3 after wounding to the date of euthanasia.

For HFD mouse model experiments, 6-wk-old male C57BL/6 mice (BioLASCO Co., Ltd., Taipei, Taiwan) were fed an HFD for rodents containing 60 kcal of fat for 10 wks. An oral glucose tolerance test (OGTT) was used to evaluate whether an HFD-induced obesity model had been established, defined as a mean 60-min OGTT value in the HFD group that was significantly higher than that in the normal diet control group (mean blood glucose level at 60 min after OGTT, 385 mg/dL). After the HFD-induced obesity model was established, mice were randomly divided into placebo or ON101-treatment groups and wounded as described above.

For M2 macrophage depletion experiments, 8-wk-old C57BL/6N mice (BioLASCO Co., Ltd.) or db/db mice were fed standard chow and water. Mice were allocated randomly to receive either m-Clodrosome (mannosylated liposomes that contain clodronate) or control m-Encapsome (Encapsula NanoSciences LLC, TN, USA) subcutaneously at a dose of 0.05 mg/kg every 2 d from 7 d before wounding until the end of the experiment. ON101-containing or placebo creams were applied topically daily from day 3 after wounding.

Images of wounds were acquired at the indicated time points, and wound areas in images were calculated using ImageJ software (National Institutes of Health, MD, USA).

### Immunohistochemical (IHC) Staining and Quantification

Full-thickness biopsy samples of skin tissue around wound edges were fixed in 10% paraformaldehyde followed by embedding in paraffin wax and sectioned at 5 μm intervals. IHC was performed (with a routine antigen-retrieval procedure) using antibodies as listed in Table 1. Whole-tissue scans were analyzed with HALO software (Indica Labs) using Area Quantification v. 1.0, Cytonuclear v. 1.5, and Cytonuclear FL v. 1.4 modules.

The quantification procedure of all IHC slides was performed using Halo software (Indica Labs) after whole slide scanning was performed using Aperio AT2 (Leica). The wound bed and surrounding tissue were first circled for quantification. For antigens that belong to membrane staining, such as CD163, CD206, CD3, MOMA2, the multiplex module was used to calculate the relative percentage of positive cells. For antibodies against cytoplasm proteins such as iNOS, MMP9, and Ly-6C/6G, an area quantification module was used to calculate the area of positive tissue/total area of the tissue of interest, expressed as a percentage. All of the quantifications were normalized with hematoxylin stain.

TABLE 1
Antibody List

| Reactive | Appli- | |
|---|---|---|
| species | cation | Antibody list (information) |
| mouse | IHC | against CD163 (ab182422, Abcam, UK), iNOS |
| | | (E-AB-70051, Elabscience, Texas, USA), CD206 |
| | | (ab64693, Abcam), cytokeratin 14 (K14; |
| | | ab181595, Abcam), Ly6G/6C (ab2557, Abcam), |
| | | MMP9 (ab228402, Abcam), CD3 (ab16669, |
| | | Abcam), MOMA2 (ab33451, Abcam) and αSMA |
| | | (ab32575, Abcam). |
| mouse | Flow | allophycocyanin (APC)-conjugated anti-mouse- |
| | | F4/80 (17-4801-82, eBioscience) and PE- |
| | | conjugated anti-mouse CD163 (12-1631-82, |
| | | eBioscience); 7-AAD (A1310, Thermo Fisher |
| | | Scientific) |
| human | Western | (p-Stat3 antibody (#9131S), Stat3 antibody |
| | blot | (#9139S), p-Akt antibody (#9271), Akt antibody |
| | | (#9272S), Cell Signaling). Proteins were detected |
| | | by enhanced chemiluminescence. |
| human | Flow | phycoerythrin (PE) CF594-conjugated anti- |
| | cytometry | hCD68 (BD Biosciences), fluorescein |
| | | isothiocyanate (FITC)-conjugated anti-hCD86 |
| | | (BioLegend), PE/Dazzle 594-conjugated anti- |
| | | hCD80 (BioLegend), Pacific Blue-conjugated |
| | | anti-hCCR7 (BioLegend), PE-conjugated anti- |
| | | hCD163 (BioLegend), BV605-conjugated anti- |
| | | hCD206 (740417, BD Bioscience), and BV510- |
| | | conjugated anti-FVS (564406, BD Biosciences). |

Quantitative Reverse Transcription Polymerase Chain Reaction (qRT-PCR)

Skin tissue was first homogenized using a TissueLyser LT (Qiagen, Hilden, Germany) with homogenization beads. Total RNA from in vitro cultured cells and skin tissues was extracted at different time points using GENEzol TriRNA Pure kits (Geneaid, Taiwan) and reverse transcribed into cDNA using First Strand cDNA Synthesis kits (Thermo Fisher Scientific, Waltham, MA, USA). qRT-PCR analyses were performed on a QuantStudio 6 Flex Real-Time PCR system using SYBR Green qRT-PCR Master Mix (both from Thermo Fisher Scientific). Primer sequences used for these experiments are listed in Table 2.

TABLE 2
Primer List

| Gene | |
|---|---|
| name | Primer sequence (5′ to 3′) |
| mIFNγ | F: CGGCACAGTCATTGAAAGCCTA |
| | (SEQ ID NO: 1) |
| | R: GTTGCTGATGGCCTGATTGTC |
| | (SEQ ID NO: 2) |
| mIL1a | F: ACGGCTGAGTTTCAGTGAGACCTT |
| | (SEQ ID NO: 3) |
| | R: AGGTGTAAGGTGCTGATCTGGGTT |
| | (SEQ ID NO: 4) |
| mCXCL1 | F: GCTTGAAGGTGTTGCCCTCAG |
| | (SEQ ID NO: 5) |
| | R: AAGCCTCGCGACCATTCTTG |
| | (SEQ ID NO: 6) |
| mCXCL11 | F: AGGAAGGTCACAGCCATAGC |
| | (SEQ ID NO: 7) |
| | R: CGATCTCTGCCATTTTGACG |
| | (SEQ ID NO: 8) |
| mIL4 | F: AGATGGATGTGCCAAACGTCCTCA |
| | (SEQ ID NO: 9) |
| | R: AATATGCGAAGCACCTTGGAAGCC |
| | (SEQ ID NO: 10) |
| mCSF3 | F: TGGCAGCAGATGGAAAACCTAG |
| | (SEQ ID NO: 11) |
| | R: AGGTACGAAATGGCCAGGACA |
| | (SEQ ID NO: 12) |
| mCXCL3 | F: AGATCTCACCACAGCCCTTC |
| | (SEQ ID NO: 13) |
| | R: AACCCTTGGTAGGGTGTTCA |
| | (SEQ ID NO: 14) |
| mGAPDH | F: CGACTTCAACAGCAACTCCCACTCTTCC |
| | (SEQ ID NO: 15) |
| | R: TGGGTGGTCCAGGGTTTCTTACTCCTT |
| | (SEQ ID NO: 16) |
| hCD80 | F: CTCACTTCTGTTCAGGTGTTATCCA |
| | (SEQ ID NO: 17) |
| | R: TCCTTTTGCCAGTAGATGCGA |
| | (SEQ ID NO: 18) |
| hCD83 | F: TCCTGAGCTGCGCCTACAG |
| | (SEQ ID NO: 19) |
| | R: GCAGGGCAAGTCCACATCTT |
| | (SEQ ID NO: 20) |
| hCD86 | F: CTGTAACTCCAGCTCTGCTCCGTA |
| | (SEQ ID NO: 21) |
| | R: GCCCATAAGTGTGCTCTGAAGTGA |
| | (SEQ ID NO: 22) |
| hCXCL9 | F: CCAACACCCCACAGAAGTGC |
| | (SEQ ID NO: 23) |
| | R: GCCAGCACCTGCTCTGAGAC |
| | (SEQ ID NO: 24) |
| hCXCL10 | F: GAACTGTACGCTGTACCTGCA |
| | (SEQ ID NO: 25) |
| | R: TTGATGGCCTTCGATTCTGGA |
| | (SEQ ID NO: 26) |
| hCCL12 | F: TCAGCCTGAGCTACAGATGC |
| | (SEQ ID NO: 27) |
| | R: CTTTAGCTTCGGGTCAATGC |
| | (SEQ ID NO: 28) |
| hCCL3 | F: TCAGACTTCAGAAGGACACGG |
| | (SEQ ID NO: 29) |
| | R: CTGCATGATTCTGAGCAGGTG |
| | (SEQ ID NO: 30) |
| hCXCL3 | F: AAGTGTGAATGTAAGGTCCCC |
| | (SEQ ID NO: 31) |
| | R: GTGCTCCCCTTGTTCAGTATC |
| | (SEQ ID NO: 32) |
| hCCL2 | F: AGGTGACTGGGGCATTGAT |
| | (SEQ ID NO: 33) |
| | R: GCCTCCAGCATGAAAGTCTC |
| | (SEQ ID NO: 34) |
| hCSF3 | F: GCTGCTTGAGCCAACTCCATA |
| | (SEQ ID NO: 35) |
| | R: GAACGCGGTACGACACCTC |
| | (SEQ ID NO: 36) |
| hGAPDH | F: ACATCGCTCAGACACCATG |
| | (SEQ ID NO: 37) |
| | R: TGTAGTTGAGGTCAATGAAGGG |
| | (SEQ ID NO: 38) |

### Detection of Mouse Skin Immune Cells by Flow Cytometry

Collected skin samples were first incubated in 0.25% Trypsin-EDTA for 30 min and subsequently 2 mg/mL collagenase at 37° C. for 1.5 h. After wash, cells were filtered by 70 and 40-μm strainers and conjugated with antibodies as listed in Table 1 in staining buffer for 30 min. Florescence activated cell sorting (FACS) analyses of stained cells were performed using a FACSAria Fusion Flow Cytometer (BD Biosciences, NJ, USA). All flow cytometry data were analyzed by FlowJo 6.0 software (BD Biosciences)

### Cell Culture and M1 and M2 Polarization

THP-1 cells were first polarized to M0 status by treatment with 60 ng/mL phorbol myristate acetate (PMA; Sigma-Aldrich, SL, USA) for 24 h and rested for an additional 24 h. For M1 polarization, cells were induced by incubating with 20 ng/mL IFN-γ (R&D Systems, MN, USA), 1 μg/mL lipopolysaccharide (LPS; Sigma-Aldrich), and 20 ng/mL GM-CSF (R&D Systems) for 48 h. For M2 polarization, M0 cells were treated with IL-4 and IL-10 (20 ng/mL; both from R&D Systems) for 48 h. For polarization procedures, cells were cultured in either high-glucose (HG) medium containing 4,500 mg/L glucose or normal-glucose (NG) medium containing 1,000 mg/L glucose as indicated in the Figure Legends.

Human ADPCs were obtained from GICC Medical, Taiwan. For gene expression analyses, ADPCs were seeded at a density of 6×10⁴ cells/mL in 12-well plates and incubated overnight. The next day, the cells were treated with 25, 50, or 100 ng/mL ON101 or vehicle control, and incubated for 24 h.

PBMCs separated from blood obtained from six healthy donors were processed for monocyte isolation as previously described (Zarif et al., Biotechniques, 2016). CD14⁺ cells were polarized into M1 or M2 macrophages by incubating with 20 ng/mL GM-CSF (R&D Systems) and M-CSF (R&D Systems) for 4 days. Thereafter, M1 macrophages were generated by treatment with 20 ng/mL IFN-γ, 1 μg/mL LPS, and 20 ng/mL GM-CSF. Different concentrations of ON101 were administered together with these cytokines for 48 h during the M1 polarization process.

### Detection of Human Macrophage Markers by Flow Cytometry

For detection of M1 and M2 macrophage markers, cultured cells were suspended then blocked by 20 μL/10⁴ of FcR Blocking Reagent (Miltenyi Biotec, Cologne, Germany). Antibodies listed in Table 1 and isotype signal for normalization were listed in Table 3 were used at 5 μL per 10⁵ cells.

TABLE 3
Baseline Value for Isotype Controls

| Antibody | Isotype | Mean | Median |
|---|---|---|---|
| CD80 | PE/CF594 | 180 | 87.8 |
| CD86 | FITC | 324 | 168 |
| CD163 | PE | 120 | 96.4 |
| CD206 | BV605 | 90.4 | 69.0 |

### Enzyme-Linked Immunosorbent Assays (ELISAs)

Culture media were collected from PBMC-derived M1 and M2 macrophages at the indicated time points. ELISA kits for TNF-α (#DY210), IL-6 (#DY206), and IL-1β (#DY201) were purchased from R&D Systems Inc., and the experiments were performed according to the manufacturer's instructions. Plates were analyzed by measuring light absorbance of wells at 450 nm using a microplate reader (Synergy H1 Hybrid Reader; BioTek, VT, USA).

### Cell Lysate Preparation and Immunoblotting

Cell lysates for immunoblotting were prepared using an M-PER mammalian protein extraction kit containing protease and phosphatase inhibitor cocktail (Thermo-Fisher Scientific). Immunoblotting using the indicated antibodies (listed in Table 1) was performed as described previously (Lu et al., J Immunother Cancer, 2020).

### Statistical Analysis

All statistical comparisons were performed using GraphPad Prism 7 (GraphPad Software, San Diego, CA). Unpaired two-tailed Student's t-tests were used to compare data sets between two independent groups, and paired Student's t-tests were used for comparing means within the same group. The difference of two groups was conducted by two-way analysis of variance (ANOVA) with Tukey's post-hoc test for multiple pairwise comparisons. Data are presented as means±standard error of the mean (SEM), and differences with a P-value <0.05 were considered statistically significant.

### Results

(i) ON101 Treatment Alters the Populations of Macrophage Subtypes and Modulates Gene Expression Profiles in Diabetic Wounds

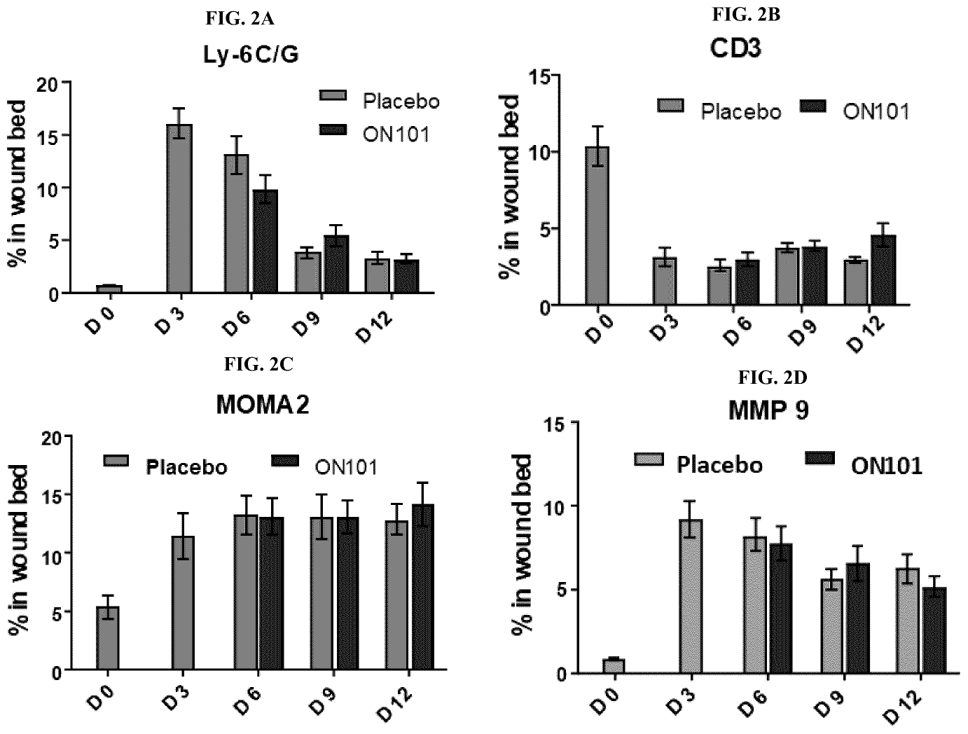

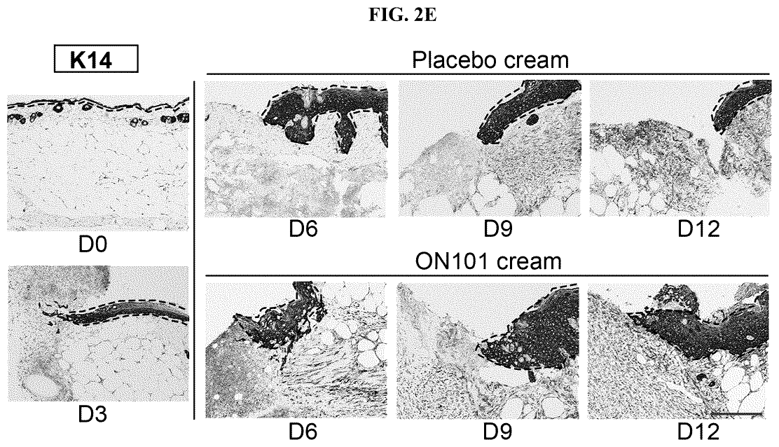

Perturbed inflammatory cues are a major cause of impaired diabetic wound healing (Eming et al., Sci Transl Med, 2014; MacLeod et al., Adv Wound Care (New Rochelle), 2016), and IHC was first performed to determine whether ON101 affected specific types of immune cells, including neutrophils (anti-Ly6G/6C), T cells (anti-CD3) and monocytes/macrophages (anti-MOMA2) (FIGS. 2A-2C). The fibroblast marker MMP9 (Liu et al., Diabetes Care, 2009) and epithelial marker keratin-14 were also analyzed (FIGS. 2D-2E). Quantifications revealed that neutrophils, fibroblasts, and T cells appeared at the first 3 days after wounding, and the expression of each marker decreased gradually during the healing process without significant difference between ON101 and placebo groups. On the other hand, total monocyte and macrophage counts increased from day 0 to day 6, with no significant differences in overall cell numbers stabilizing from day 6 to 12 (FIG. 2C) between the two groups.

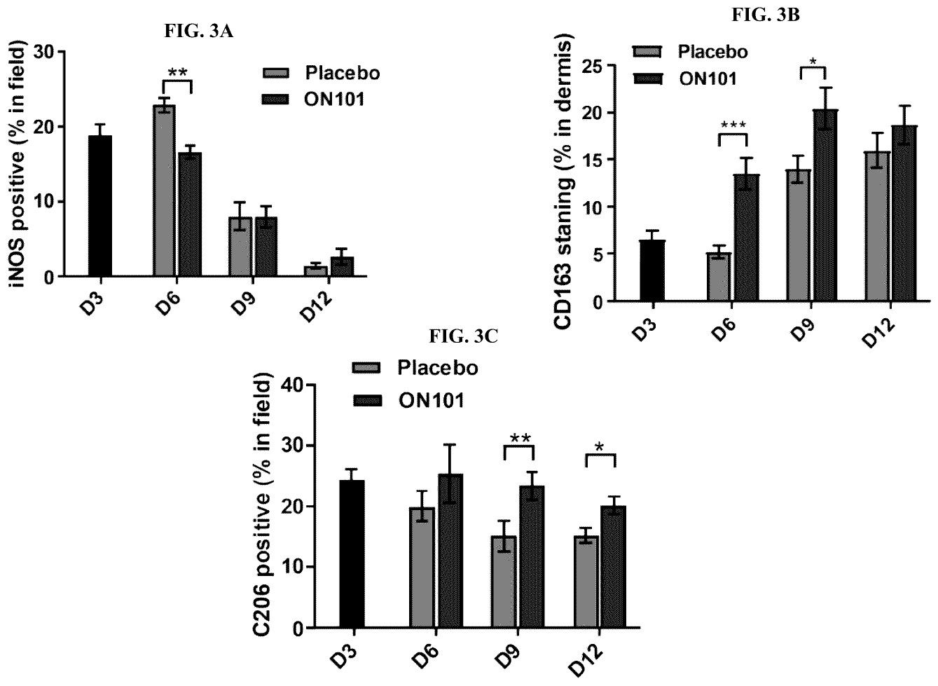

Among monocytes and macrophages, the inducible nitric oxide synthase (iNOS)-positive M1 subtype of macrophages decreased over time (FIG. 3A). Specifically, the proportion of iNOS⁺ cells was reduced significantly around wound edges in the ON101 group on day 6 after wounding compared with the placebo group (FIG. 3A). The increased number of CD163⁺ cells was observed on day 6 (FIG. 3B), whereas the expression pattern of CD206⁺ cells marked enriched after wounding on day 3 (FIG. 3C), indicating earlier CD206⁺ enrichment than CD163⁺. In addition, the proportions of CD163⁺ and CD206⁺ cells were increased from day 6 in ON101 treatment wounds when compared with placebo groups (FIGS. 3B and 3C). The iNOS-MOMA2 or CD163⁻ MOMA2 double staining images showed that a decreased number of iNOS⁺MOMA⁺ double positive staining in the ON101 treatment group and a greater number of CD163⁺ MOMA2⁺ could be observed in the ON101 treatment group when compared with their corresponding Placebo controls. These results imply that ON101 treatment might modulate the proportion of M1 and M2 macrophages around the diabetic wound bed.

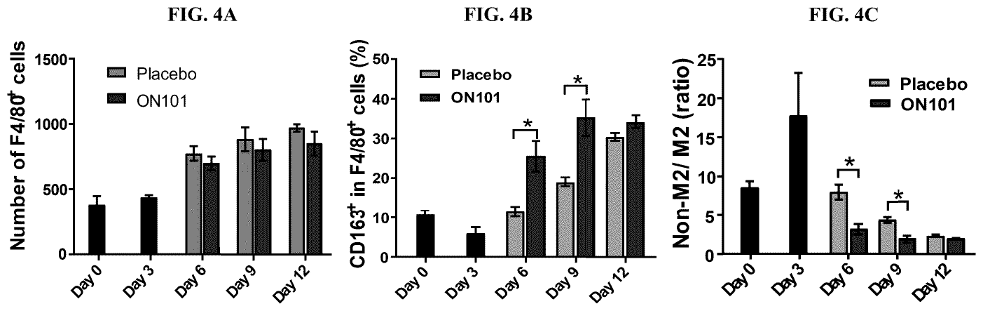

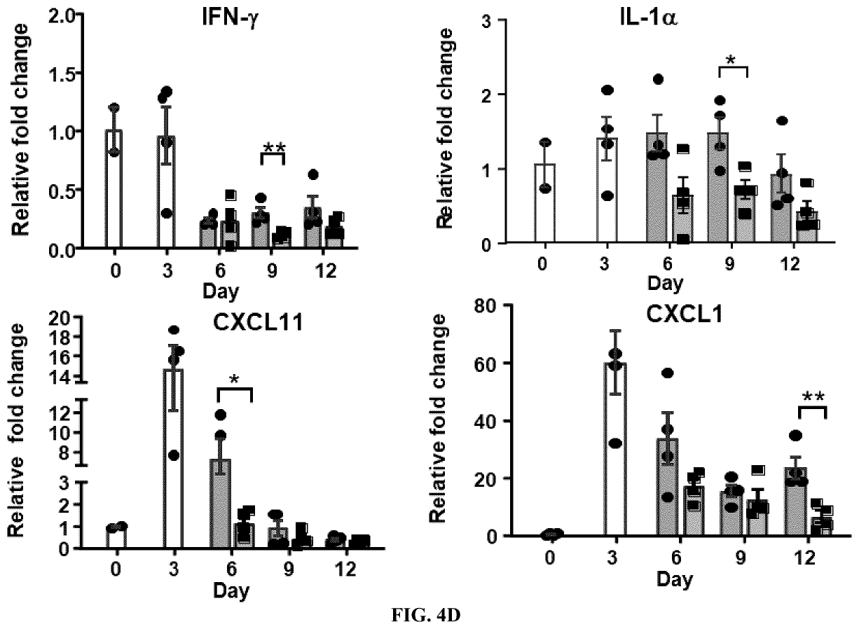

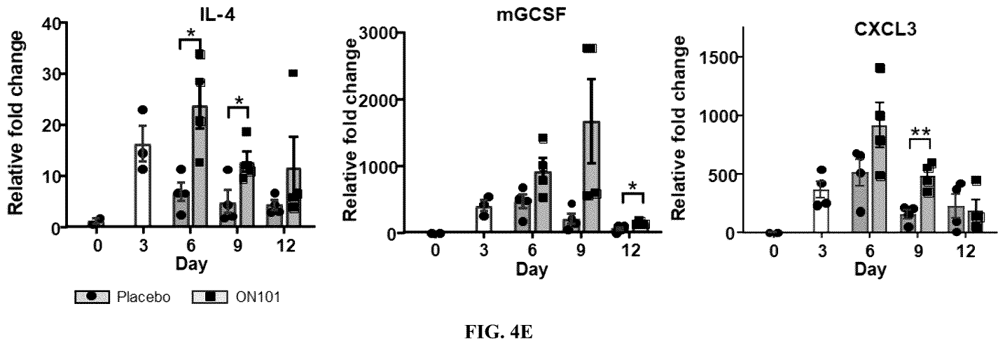

To quantify the dynamic changes in macrophage subtypes induced by ON101, macrophages isolated from wound-surrounding tissues of db/db mice treated with either ON101 or placebo cream were analyzed by flow cytometry. These analyses confirmed a gradual increase of total macrophages (F4/80⁺) around the wound but without significant differences between groups (FIG. 4A). Notably, the proportion of CD163⁺ cells among all macrophages (CD163⁺ in F4/80⁺ cells) was significantly increased in the ON101 treatment group compared with the placebo group (FIG. 4B), indicating enhanced recruitment or increased numbers of M2c-like macrophages in the ON101 treatment group. In addition, a lower non-M2 cells/M2 ratio was observed in the ON101 treatment group on days 6 and day 9 compared with the placebo group (FIG. 4D), suggesting that ON101 may modulate the population of macrophage subtypes.

Next, a study was performed to explore which genes might be affected by ON101 in diabetic wounds. 86 wound healing-related genes were analyzed using a TaqMan Array Mouse Wound Healing Panel. Confirmed by qRT-PCR, several M1-associated genes, including IFN-γ, IL-1α, CXCL1, and CXCL11, were downregulated, whereas the M2-associated gene IL-4 was upregulated by ON101 treatment (FIGS. 4D-4E). In addition, genes likely involved in lymphocyte recruitment and differentiation, such as CXCL3 and GCSF (Martin et al., Semin Immunol, 2021, Reyes et al., Adv Exp Med Biol, 2021), were also upregulated by ON101 treatment (FIG. 4E). These results suggested that topical application of ON101 downregulates pro-inflammation-associated genes and upregulates M2-associated gene and other genes related to chemoattraction or cell differentiation in a diabetic wound.

(ii) ON101 Directly Suppresses M1 Macrophage Polarizing and Restores CD206⁺ M2 Macrophages by Reversing M1-Mediated M2 Suppression

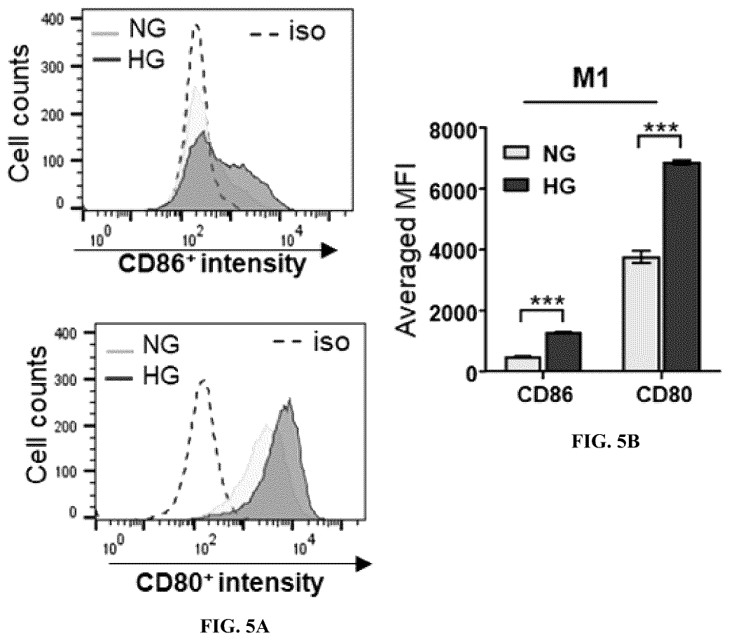

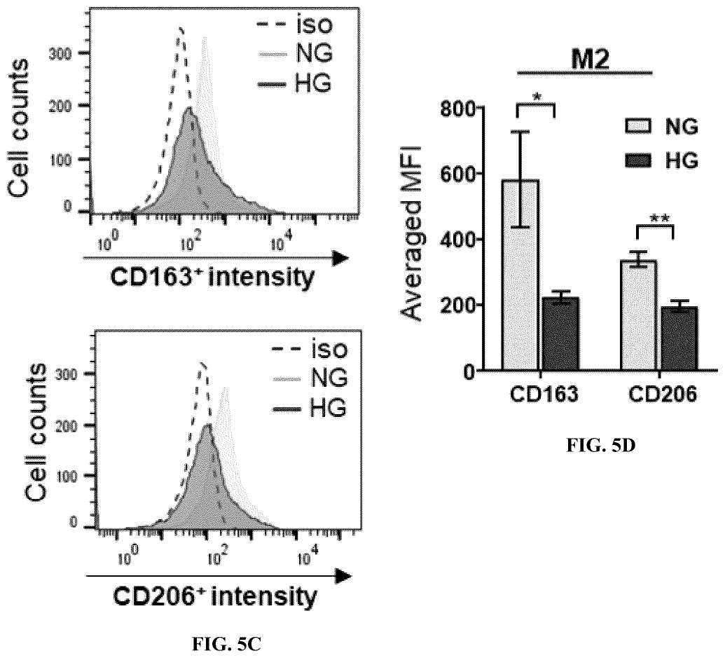

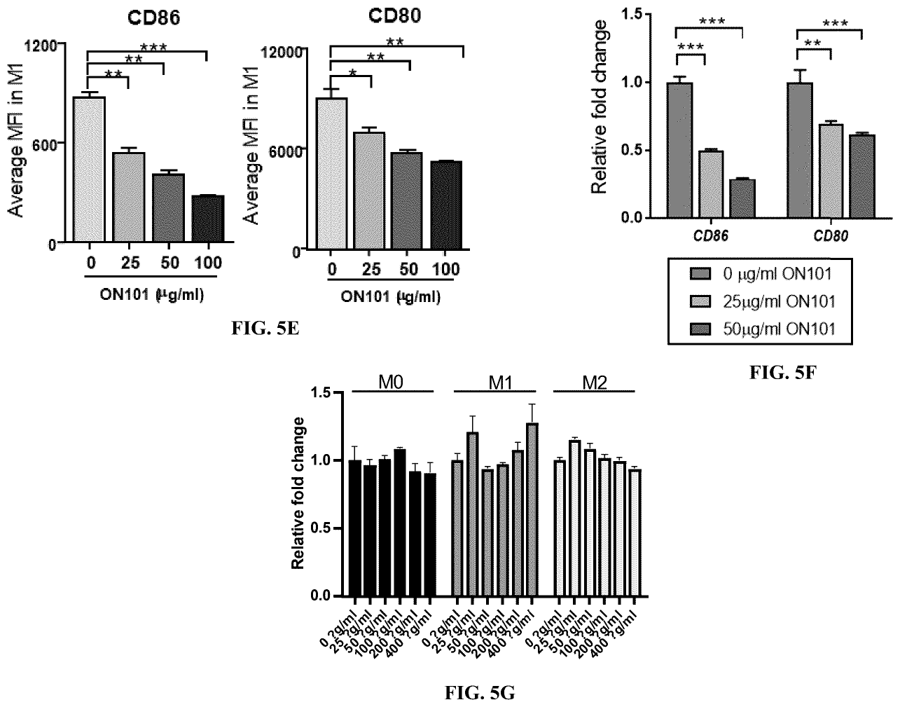

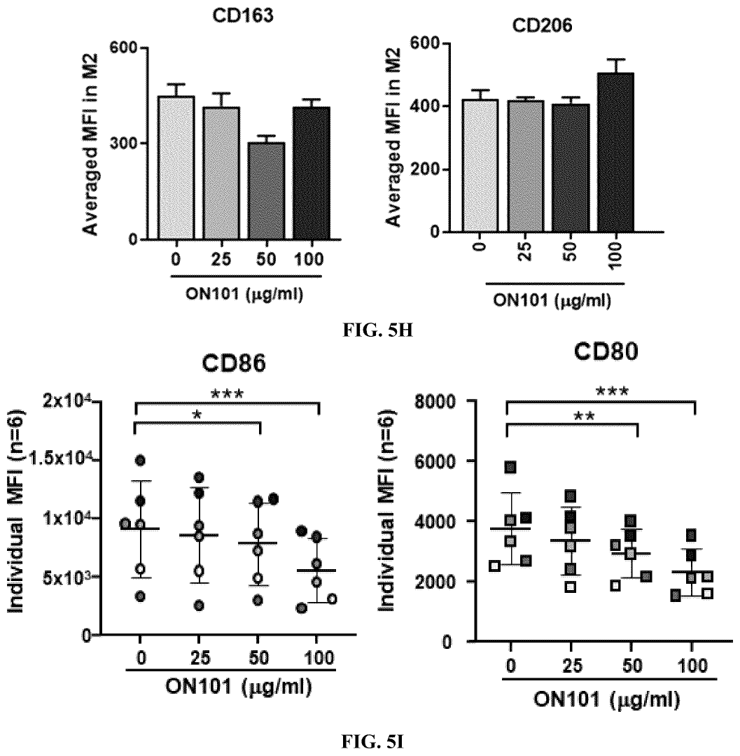

Poor glycemic control in patients with diabetes is often associated with chronic inflammation and elevated levels of pro-inflammatory cytokines (Chang et al., Crit Rev Oncol Hematol, 2016). To test the M1 or M2 polarization under conditions described herein, the polarizable human monocyte cell line, THP-1, was used to polarize M1 or M2 macrophages under either normal glucose (NG) or high-glucose (HG) conditions. The results demonstrated that the proportion of CD86 and CD80 (M1 markers) was higher among cells cultured in HG compared with NG conditions (FIGS. 5A-5B). In contrast, the proportion of M2a/c markers (CD163⁺ and/or CD206⁺) following M2-polarizing conditions was lower in HG medium than the NG medium (FIGS. 5C-5D).

After establishing this model and under the same experimental conditions, increasing concentrations of ON101 were cultured with the cells in the M1-polarizing condition, which led to a dose-dependent suppression in the proportion of CD86- and CD80-positive M1 macrophages (FIG. 5E). This was not attributable to nonspecific cytotoxic effects (FIG. 5G). CD80 and CD86 mRNA levels were also downregulated by ON101 (FIG. 5F), suggesting that ON101 transcriptionally suppressed CD80 and CD86. In contrast, in the in vitro M2 macrophage polarization model, ON101 did not affect expression levels of CD163 or CD206, suggesting that ON101 does not directly affect M2 polarization (FIG. 5H).

Ex vivo M1 polarization experiments isolated from human peripheral blood mononuclear cells (PBMCs) further revealed that, although basal amounts of CD14⁺/CD68⁺/CD86⁺ and CD14⁺/CD68⁺/CD80⁺ cells differed among six donors, proportions of CD86⁺ and CD80⁺ levels appeared to be suppressed in a concentration-dependent manner by co-administering ON101 in HG medium (FIG. 5I). Collectively, these findings demonstrated that ON101 directly inhibits M1 polarization by suppressing the expressions of CD80 and CD86.

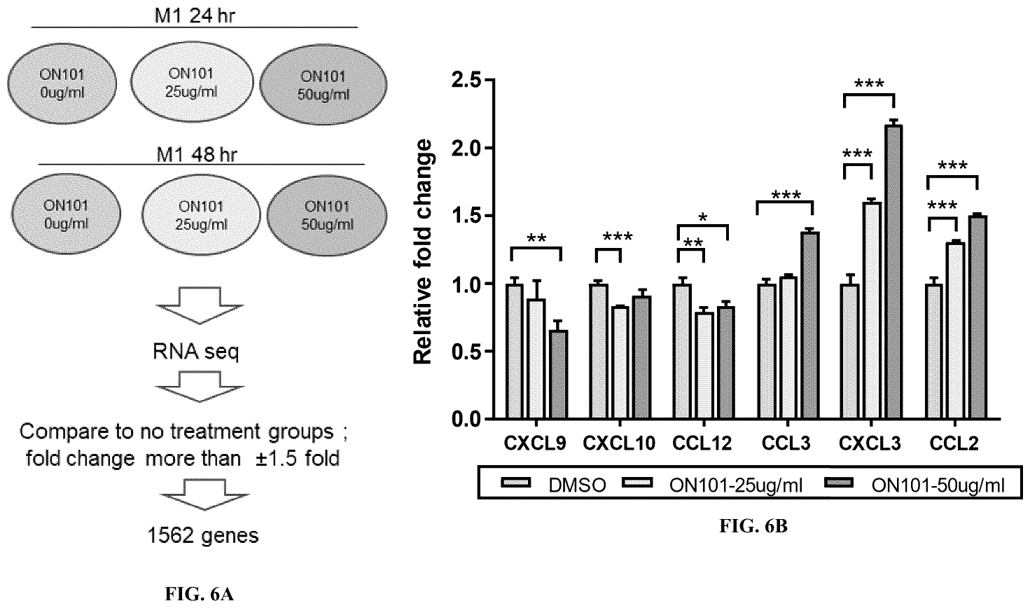

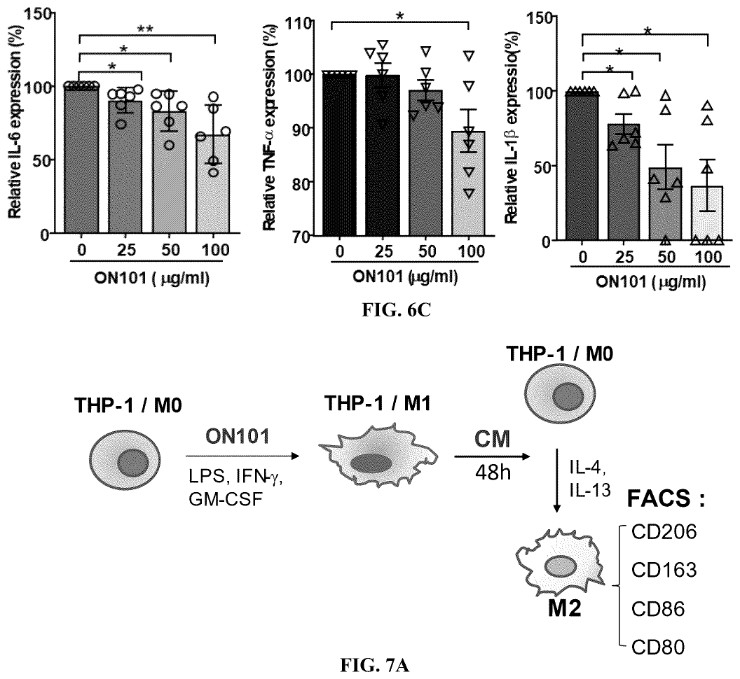

To further explore whether ON101 alters M1 macrophage functions, RNA sequencing was performed (FIG. 6A). A total of 121 genes changed following ON101 treatment. qRT-PCR validation showed that ON101 downregulated the M1-associated chemokines CXCL9, CXCL10, and CCL12, which function in Th1-mediated immune activation (Kuo et al., Front Med (Lausanne), 2018) (FIG. 6B). CCL2 and CCL3, cytokines involved in monocyte and/or macrophage recruitment and migration (Schraufstatter et al., Immunology, 2012, Zhuang et al., J Dent Res, 2019), were significantly upregulated by ON101 treatment (FIG. 6B). In addition, an examination of pro-inflammatory cytokines released from ex vivo polarized M1 macrophages from six independent donors revealed that ON101 significantly suppressed levels of IL-6, IL-1β, and TNF-α (FIG. 6C). These findings demonstrated that ON101 treatment alleviates M1-associated inflammation and provides an environment that favors monocyte recruitment.

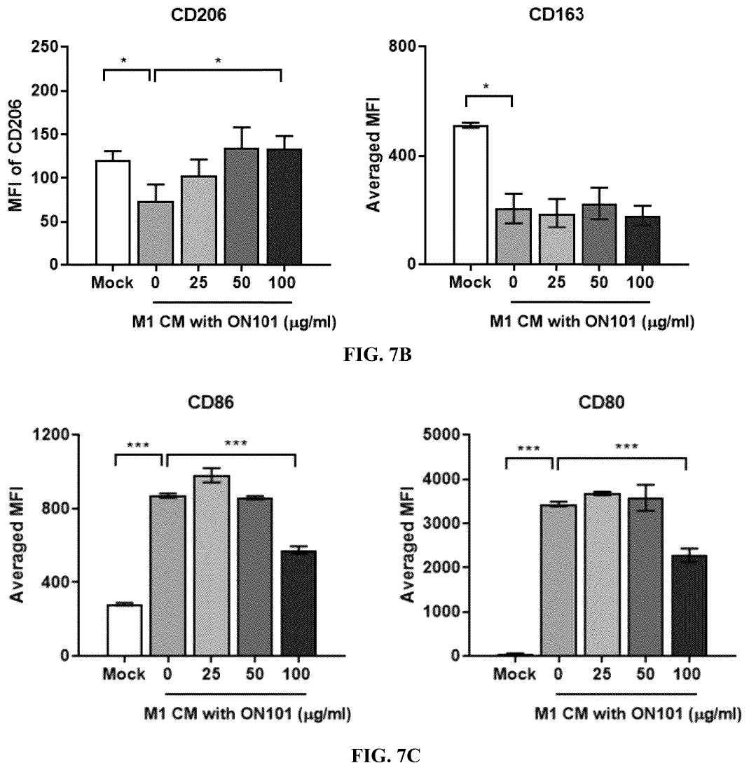

To determine whether M1-dominated pro-inflammatory cues impair the polarization of M2 macrophages, conditioned medium was collected from M1 polarization cultures (M1-CM) and co-treated with M2 polarizing agents to see the extent of M2 polarization as shown in FIG. 7A. Flow cytometry analyses demonstrated that M1-CM dramatically suppressed the expression of both CD206 and CD163 during the M2 polarization process (FIG. 7B). By contrast, ON101 treated M1-CM significantly rescued the levels of CD206-positive macrophages but not CD163 (FIG. 7B). In addition, M1-CM increased CD80 and CD86 levels in M2 macrophages, whereas ON101 treated M1-CM downregulated both CD86 and CD80 expression (FIG. 7C). These results revealed the repressive role of factors secreted from M1 macrophages to counter a microenvironment favoring M2 polarization. Thus, ON101 is likely to function in mitigating the M1-dominant milieu, changing it to an M2a-favorable environment.

(iii) ON101 Enhances the Activity of Adipocyte Progenitor Cells (ADPCs) to Promote M1-to-M2 Transition

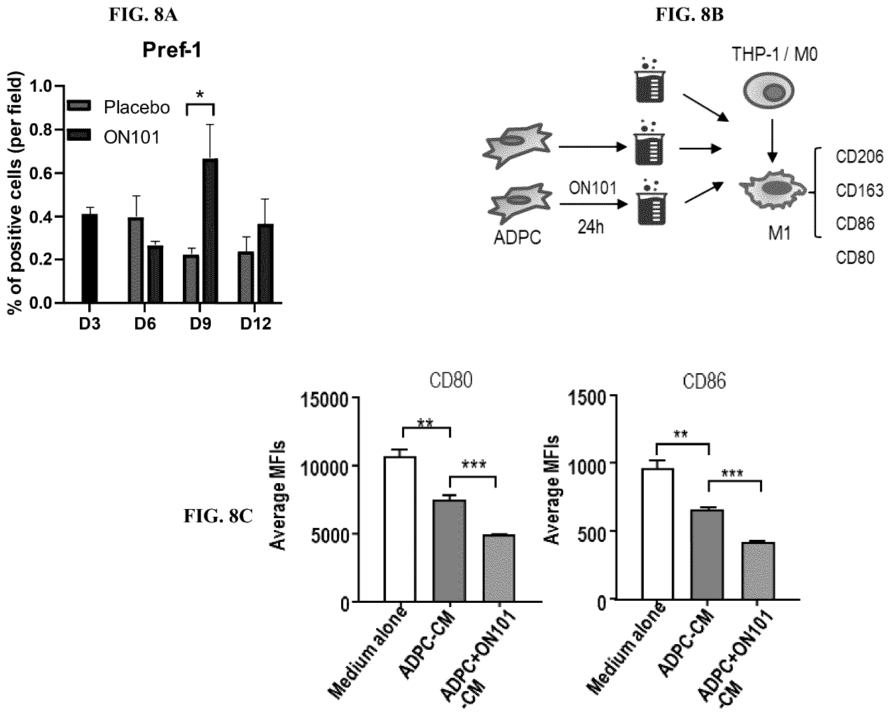

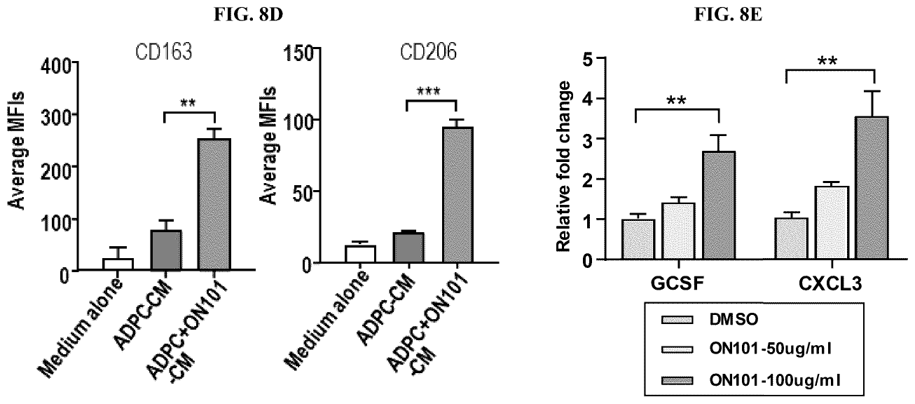

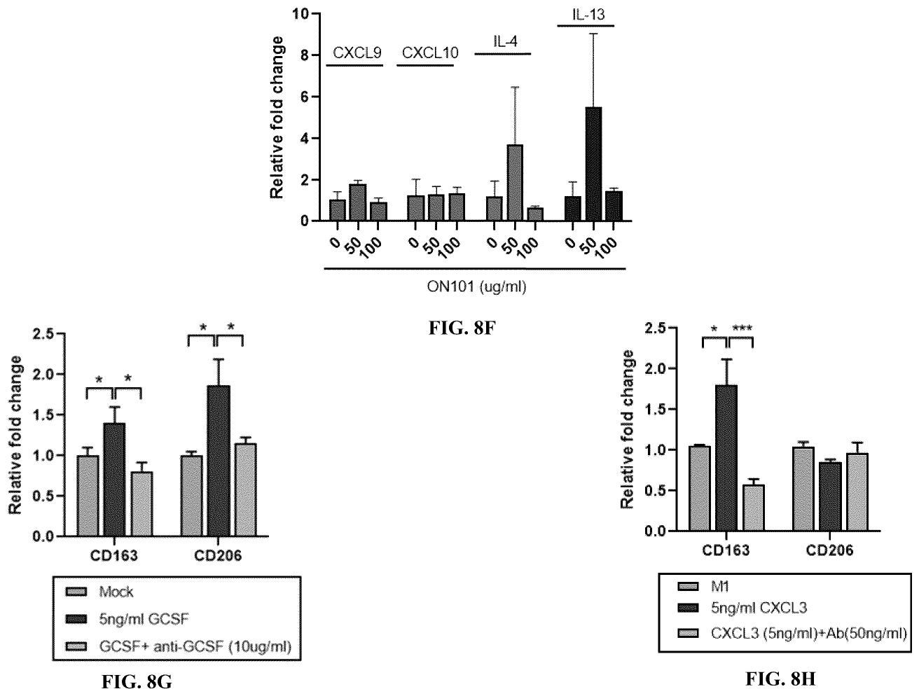

ADPCs, identified by their positive staining for Pref-1 (pre-adipocyte factor-1), are skin-resident mesenchymal stem cells with multiple regenerative potentials that mediate skin regeneration and diabetic wound repair (Gadelkarim et al., Biomed Pharmacother, 2018; Sul, Mol Endocrinol, 2009). There was a noticeable increase in Pref-1-positive cells around the subcutaneous fat layer during the wound healing process in ON101-treated db/db mice compared with mice in the placebo group (FIG. 8A), suggesting that ON101 might activate ADPCs.

Because ON101 treatment increased the expression of CD163⁺ cells during the healing process in diabetic mice, further studies were performed to evaluate whether ON101 promoted CD163⁺ M2 polarization through activation of adipocyte stem cells. To test this, CM from cultures of ADPCs (ADPC-CM), with or without ON101 treatment was applied to the M1 polarization assay (FIG. 8B). ADPC-CM significantly suppressed CD80 and CD86 (FIG. 8C) but only slightly increased the intensity of CD163 and CD206 (FIG. 8D). Interestingly, the application of ON101-treated ADPC-CM significantly increased the intensities of both CD206 and CD163 markers (FIG. 8D), suggesting that ADPCs play a significant role in ON101-induced promotion of the M1-to-M2 transition.

(iv) ON101 Stimulates ADPC Expression of GCSF and CXCL3, which are Engaged in the M1-to-M2 Transition

Next, experiments were performed to analyze whether the genes altered by ON101 treatment in diabetic wounds could be produced by ADPCs upon ON101 treatment and focused on candidate genes detected in wound tissues in ON101-treated db/db mice (FIGS. 4E-4F). The qRT-PCR results demonstrated that ON101 induced a concentration-dependent increased expression of GCSF and CXCL3 (FIG. 8E) but not other genes in ADPCs (FIG. 8F). To test whether GCSF and CXCL3 might be involved in the marker switch between M1 and M2 macrophages, recombinant GCSF and CXCL3 proteins, with or without their neutralizing antibodies, were co-administrated with M1-polarizing cytokines. GCSF treatment caused a concentration-dependent increase in both CD163 and CD206 (FIG. 8G), whereas CXCL3 treatment only induced CD163 expression (FIG. 8H). Both GCSF- and CXCL3-mediated inductions of M2a/c markers were abolished by the corresponding neutralizing antibodies (FIGS. 8G-8H). In addition, both GCSF and CXCL3 treatments activated Stat3 signaling pathways, whereas GCSF activated Akt phosphorylation, which was again abolished by their corresponding antibodies (FIG. 8I). These results revealed ON101 can readily stimulate both GCSF and CXCL3 gene expression in ADPCs, and their expressions may subsequently promote the activation of CD206⁺ and/or CD163⁺ M2 subtypes in M1 macrophages.

(v) M2 Macrophages are Critical for ON01-Accelerated Wound Healing

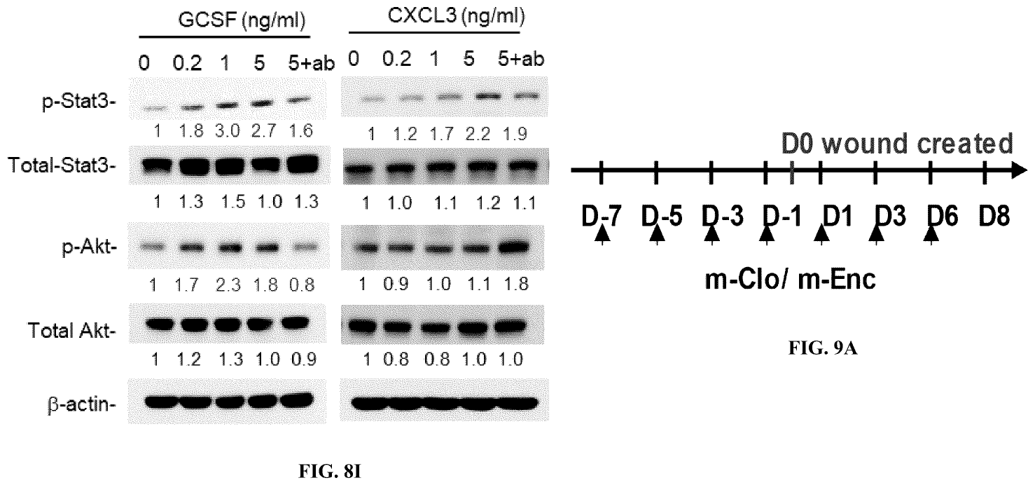

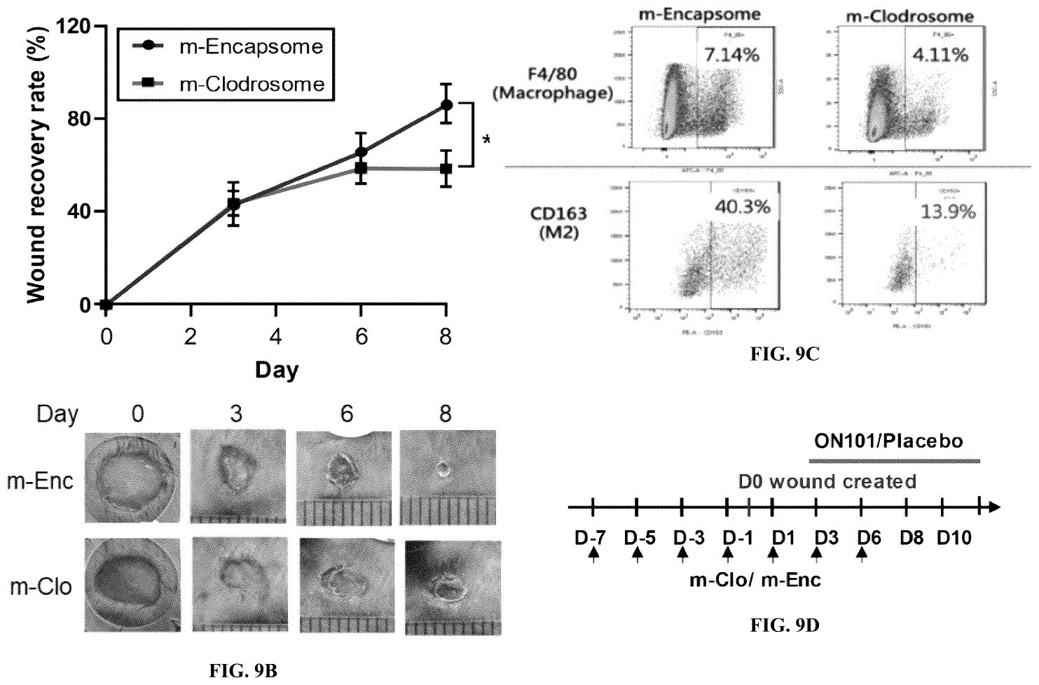

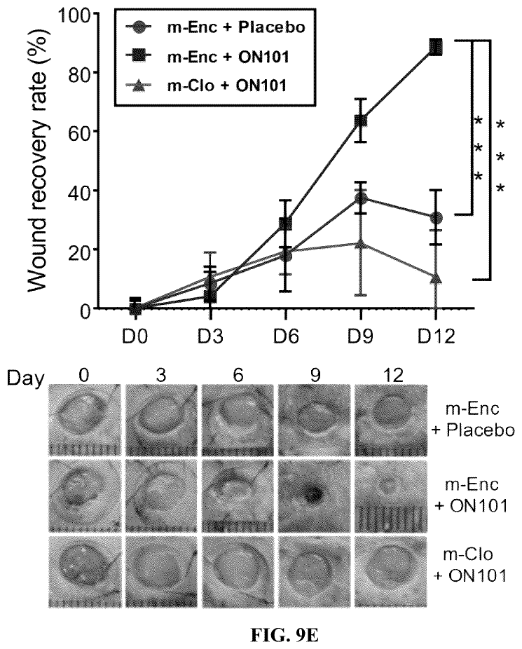

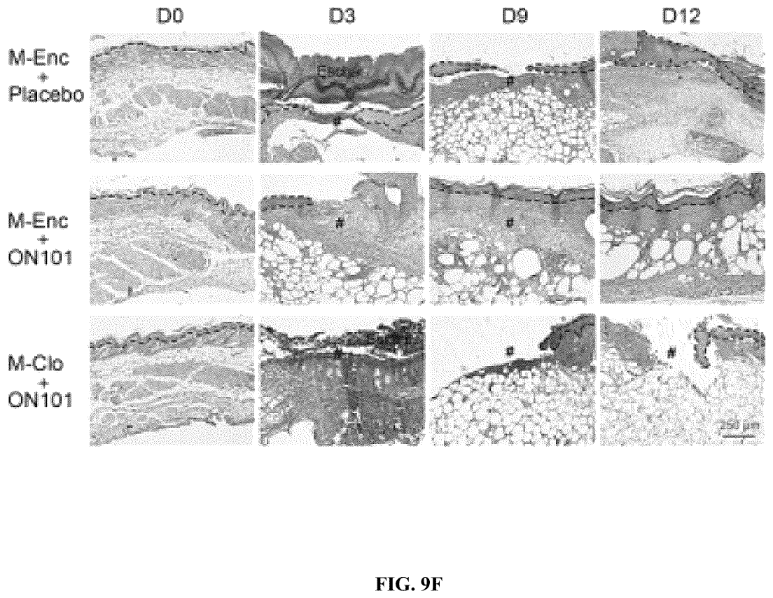

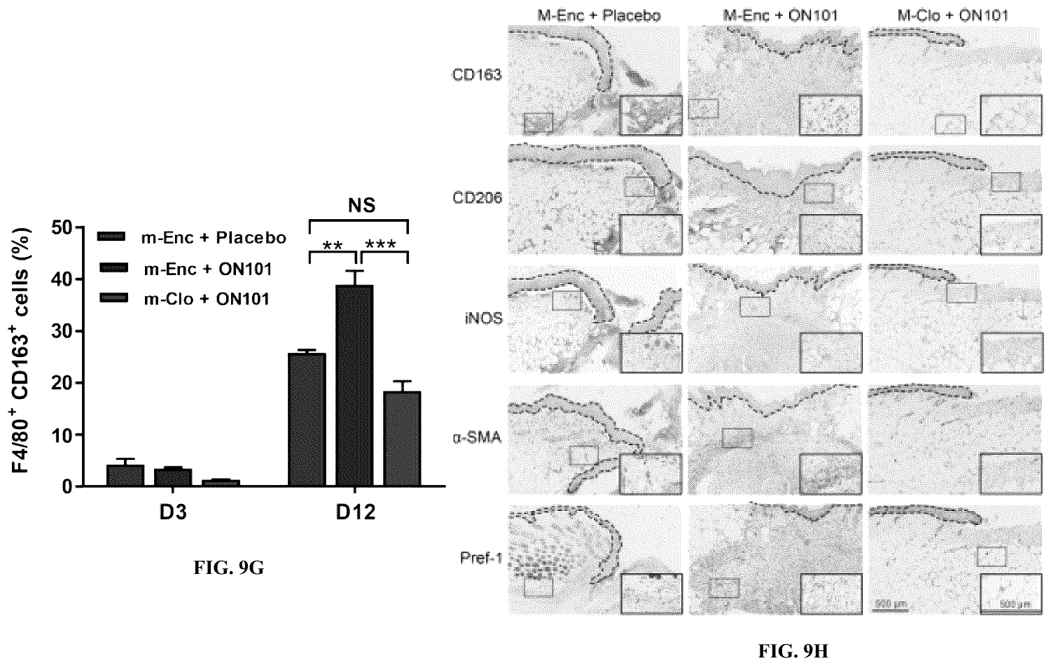

Because ON101 exerts dual effects on M1/M2 ratios, downregulating M1 and promoting M2 macrophages, experiments were performed to clarify if M2 macrophages are critical for normal wound healing by a loss-of-function assay of M2 macrophages. Mannosylated clodronate (m-clodronate; m-Clo) was applied to deplete M2 macrophages (Chu et al., Nat Commun, 2019). Mice were pretreated with mannosylated clodronate or control liposomes (m-Encapsomes; m-Enc) for 7 days before wounding, and then continue treatment for 6 days after wounding (FIG. 9A). CD163⁺ M2 macrophages were efficiently depleted by m-Clo as determined by flow cytometry (FIG. 9C). Compared to the m-Enc group, wound healing was delayed in the m-Clo group, suggestive of the vital role of M2 macrophages in normal wound healing. (FIG. 9B).

Further testing sought to evaluate whether ON101 treatment can promote diabetic wound healing by enriching the population of M2 macrophages. Following the administration of m-Clo or m-Enc, db/db mice were co-treated with ON101 or placebo cream after wounding (FIG. 9D). ON101 treatment improved the wound healing rate compared with the placebo group; notably, this effect was abolished by reducing the proportion of M2 macrophages through m-Clo treatment (FIGS. 9E-9F). Flow cytometry analysis of M2 macrophages further indicated that ON101-accelerated wound healing was accompanied by an increase in the population of CD163⁺ macrophages, an effect that was also effectively attenuated by m-Clo treatment (FIG. 9G). IHC staining demonstrated a thin dermis layer and a deficiency of CD163⁺ and CD206⁺ cells around the wound bed in the m-Clo treatment group (FIG. 9H). In addition, the extracellular matrix remodeling protein, smooth muscle alpha actin (α-SMA), as well as Pref-1⁺ cells, were markedly increased in the ON101 treatment group, effects that were attenuated by M2 depletion (FIG. 9H). On the basis of these results, topical ON101 treatment promoted diabetic wound healing through dual pathways-attenuated M1-mediated pro-inflammatory cues and enhanced M2 macrophage populations—by enabling the M1-to-M2 transition via factors secreted by ADPCs. Thus, ON101 facilitates diabetic wound healing via an M2-macrophage-dependent pathway.

### Example 2. Immune Suppressive Activities of Plectranthus amboinicus (PA) Extracts

This example illustrates that Plectranthus amboinicus (PA) extract, optionally in combination with Centella asiatica (CA) extract, exhibited immune suppressive activities, indicating that the PA extract, taking alone or in combination with the CA extract as disclosed herein would benefit treatment of autoimmune diseases.

Briefly, the ON101 topical cream disclosed herein was investigated a range of activities in modulating the development of autoimmune responses and diseases, such as alleviating psoriatic symptoms, suppressing pathways involved in immune activation, up-regulating skin barrier-related gene expressions and keratinocyte differentiation markers, up-regulating pathways involved in attenuating autoimmune responses, and suppressing dendritic cell activation and maturation.

(i) PA-F4 Alleviates Imiquimod-Induced Psoriatic Symptoms

Imiquimod (IMQ) is an immune modulator which induces psoriasis symptoms and has been widely used in the research of psoriasis especially in a mouse model (van der Fits et al., J Immunol, 2009).

In this experiment, a 5% IMQ cream was applied topically daily from day 1 to day 6 to induce psoriasis symptoms in mice. Testing treatments included the tapinarof cream, a 0.5% PA-F4 cream, and a 1% PA-F4 cream. Tapinarof, also known as benvitimod, is a medication used as a topical treatment for plaque psoriasis.

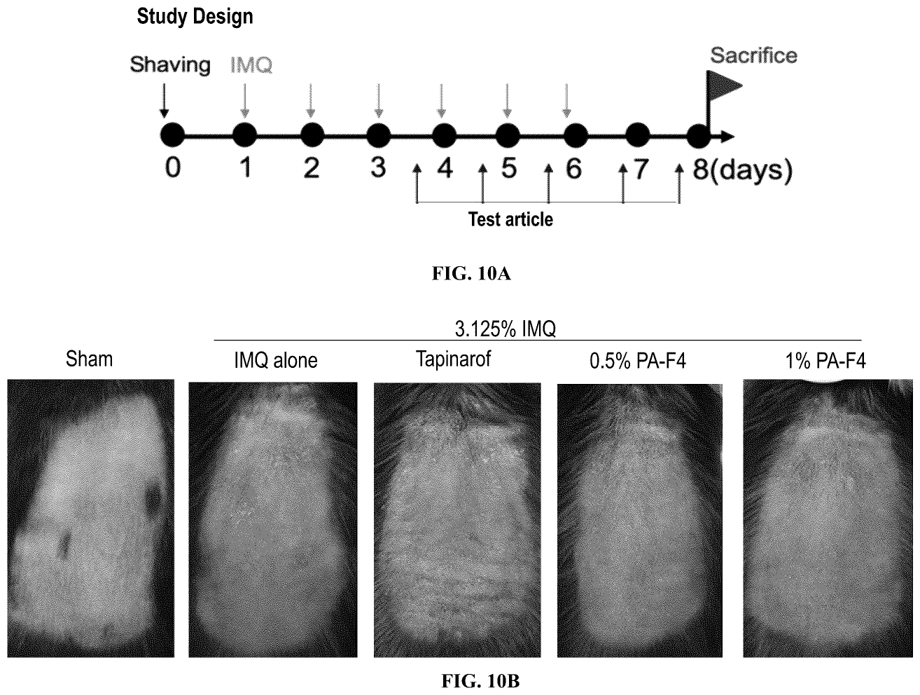

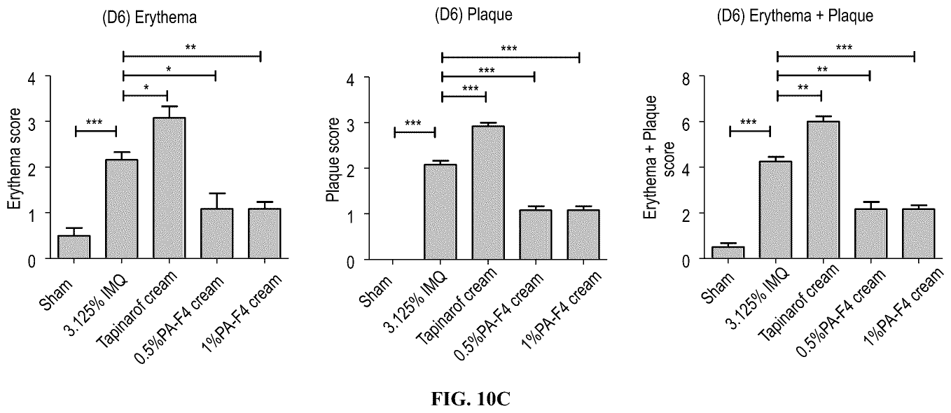

Treatments were applied topically to mice daily from day 3 to day 7, and 6 hours after IMQ application if IMQ were to be applied on the same day. The severities of erythema and plaque were scored at day 6 by three independent evaluators. A scheme of the experimental design is shown in FIG. 10A (timeline) and Table 4 (treatment groups).

TABLE 4
Study Design for Testing PA-F4 in
Alleviating IMQ-Induced Psoriasis

| Group | Test article | Animal No. |
|---|---|---|
| 1 | Sham control | 4 |
| 2 | IMQ alone | 4 |
| 3 | IMQ + Tapinarof cream | 4 |
| 4 | IMQ + 0.5% PA-F4 cream | 4 |
| 5 | IMQ + 1% PA-F4 cream | 4 |

As shown in FIGS. 10B-10C; both the PA-F4 topical formulations alleviated IMQ-induced psoriasis symptoms, including erythema and plaque, in levels greater than the Tapinarof cream. The results are statistically significant.

Taken together, this experiment illustrates the use of PA-F4 in alleviating psoriatic symptoms using a mouse model, providing insight into treatment options for autoimmune diseases such as psoriasis.

(ii) PA-F4 Suppresses IL-17/IL-23 Pathways in IMQ-Induced Skin Tissue in a Dose Dependent Manner

The pathogenesis of psoriasis has been suggested to be linked to the pathogenesis of chronic inflammatory diseases with a dominant IL-23/Th17 axis (Rendon et al., Int J Mol Sci, 2019). Atopic dermatitis (AD) is a heterogenous disorder that can be classified into different types. In intrinsic AD, the skin barrier is relatively preserved and metals or haptens can penetrate through the skin. Type 1 and Th17 (IL-17A, CCL20, Elafin, and IL-22) reactions occur in addition to Type 2 responses, which may share a same feature with the Asian-type AD (Tokura et al., Allergol Int, 2022).

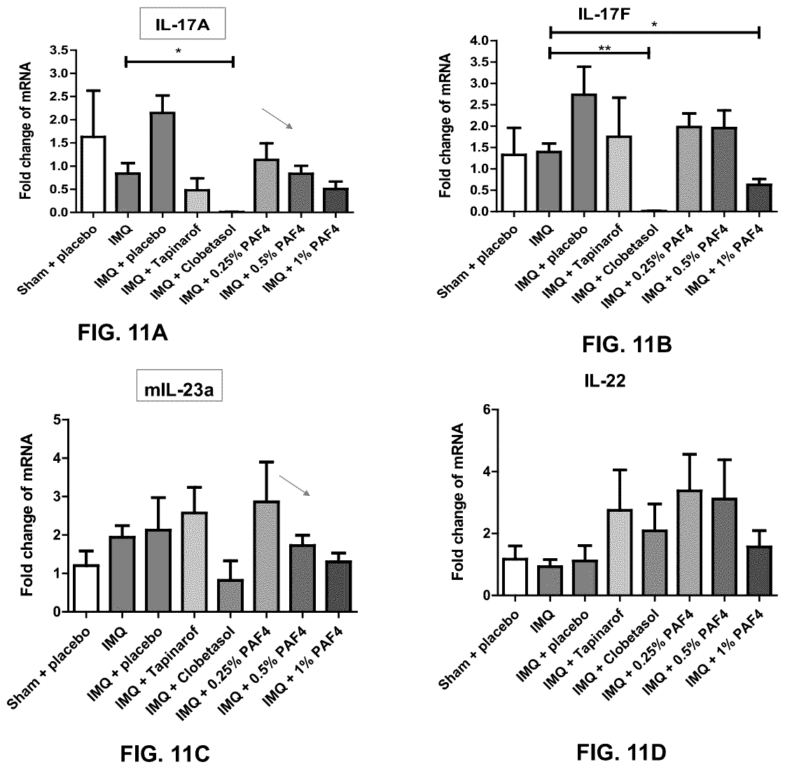

The results obtained from this study show that the expression levels of IL-17a, IL-17f, and IL-23 were suppressed by PA-F4 in a dose dependent manner, suggesting that PA-F4 could inhibit the secretion of these cytokines on the psoriasis or AD lesions and down-regulate the IL-17 and IL-23-mediated immune activation. FIGS. 11A-11D.

Taken together, this experiment illustrates that PA-F4 dose dependently suppressed IL-17/IL-23 pathways in IMQ-induced skin tissue, providing insight into treatment options for autoimmune diseases such as psoriasis and atopic dermatitis (e.g., intrinsic atopic dermatitis).

(iii) PA-F4 Up-Regulates Skin Barrier Related Gene Expressions

The complex compositions and functions of the skin barrier play an important role in the initiation and maintenance of skin inflammation. Profound alterations in skin barrier functions, epidermal morphology, and stratum corneum (SC) lipid compositions have been well characterized in atopic dermatitis (AD).

Defects in skin barrier genes have been shown to play a key role in the development of allergic diseases, including increased risk of developing AD alongside asthma, allergic rhinitis, food allergy, and hay fever (Luger et al., J Dermatol Sci, 2021; Tsakok et al., Br J Dermatol, 2019; Kelleher et al., J Allergy Clin Immunol, 2016)

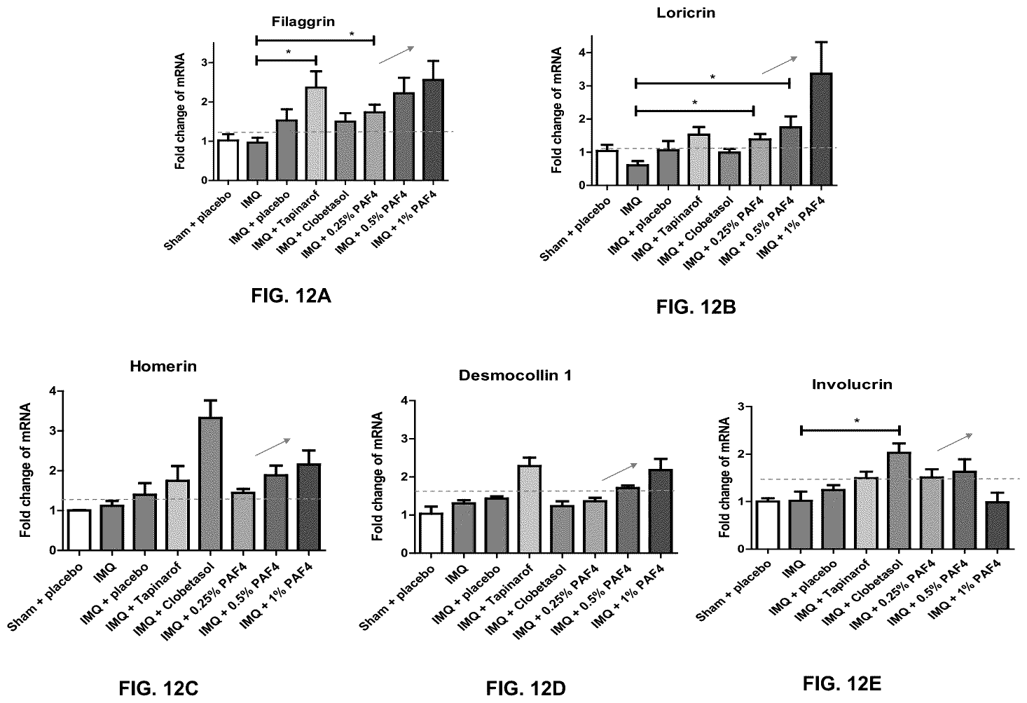

As shown in FIGS. 12A-12E, the PA-F4 formulation up-related expression levels of filaggrin, loricrin, homerin, desmocollin, and involucrin in a dose-dependent manner, suggesting that PA-F4 application could strengthen skin barrier functions.

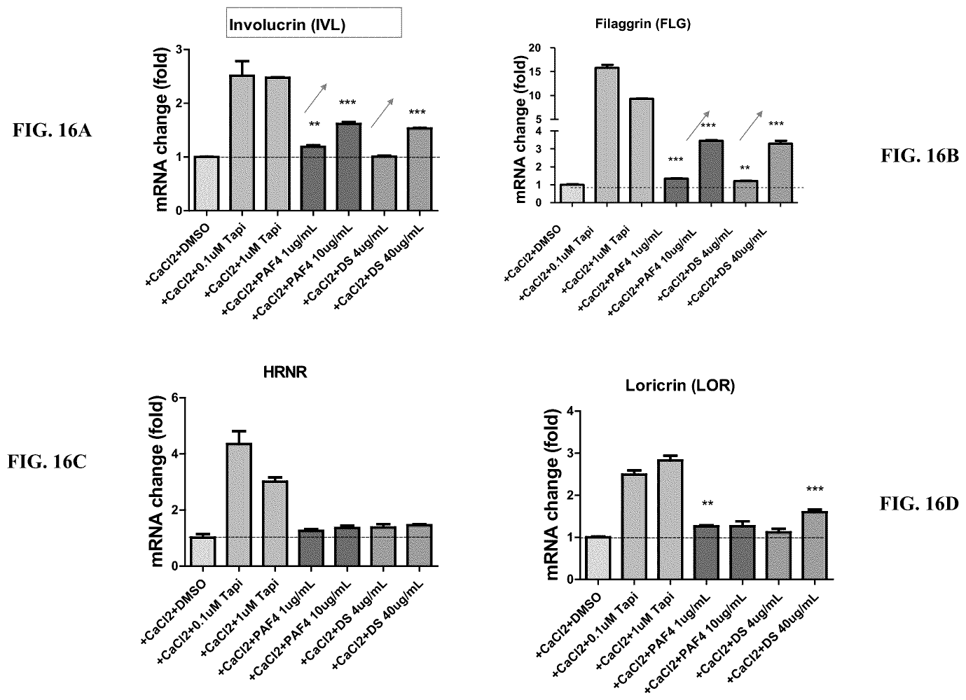

Additionally, adult human epidermal keratinocytes were treated by compositions having (a) 1.2 mM CaCl2) and (b) the PA-F4 extract, Tapinarof, or DS for 72 hours and the treated cells were subjected to RT-PCR to analyze expression changes of Involucrin, Filaggrin, HRNR, and Loricrin. As shown in FIGS. 16A-16A, PA-F4 also updates expression of involucrin.

Taken together, this experiment illustrates that PA-F4 dose dependently suppressed IL-17/IL-23 pathways in IMQ-induced skin tissue, providing insight into treatment options for autoimmune diseases such as extrinsic atopic dermatitis, asthma, allergic rhinitis, food allergy, and hay fever.

(iv) PA-F4 Up-Regulates Keratinocyte Differentiation Markers

Keratinocytes are cells that make up over 90% of the epidermis or the outer layer of the skin. Inside the epidermis, keratinocytes are arranged in four different layers the stratum basale, stratum spinosum, stratum granulosum, and stratum corneum. Differentiation of keratinocytes is critical for epidermal stratification and formation of a protective stratum corneum, which involves a series of complex processes leading through gradual changes in characteristics and functions of keratinocytes up to their programmed cell death via cornification. Keratinocytes produce keratins, also known as intermediate filament proteins, which hold the skin cells and layers together. Differentiation of cells from the basal to the spinous layer is characterized by a shift in keratin production from keratins 5 and 14 to keratins 1 and 10. Thus, keratin 1 and keratin 10 (K1 and K10) are markers associated with keratinocyte differentiation (Xiao et al., Chin Med J (Engl), 2020). Previous research has shown that expression levels of the genes encoding K1 and K10 were significantly down regulated in psoriatic skin (Jiang et al., Exp Cell Res, 2017).

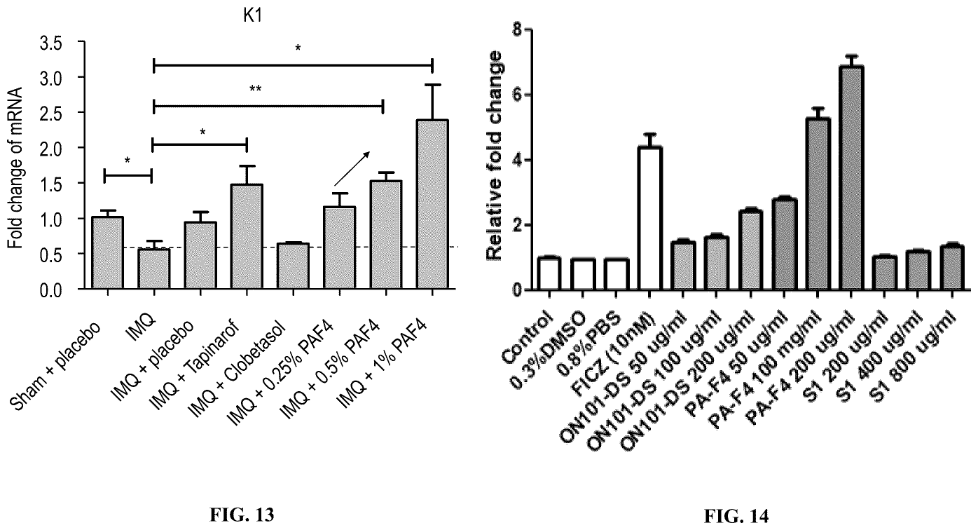

Results of this experiment are shown in FIG. 13, which illustrates that expression of K1 was up-regulated by PA-F4 in a dose-dependent manner. This suggests that PA-F4 could promote keratinocyte differentiation.

Taken together, this experiment illustrates that PA-F4 up-regulates expression of keratinocyte differentiation marker, providing insight into treatment options for autoimmune diseases such as psoriasis.

(v) PA-F4 and ON101 Dose Dependently Up-Regulate AhR Signaling Pathway

The aryl hydrocarbon receptor (AhR) was initially identified in early toxicology studies that observed an increase in monooxygenase activity after exposure polycyclic aromatic hydrocarbons, which was involved in the metabolism of such environmental chemicals. The AhR gene has been revealed as highly conserved throughout evolution, and the fact that the AhR gene has been so highly conserved provides evidence of the fundamental importance of the AhR in biological systems. Current evidence also supports that the AhR plays an important role within biological systems across several species. In the mouse, for instance, targeted disruption of the AhR results in incomplete development of immune organs and altered immune function. Recent studies have shown that the AhR signaling pathway attenuates autoimmune responses. For instance, it has been shown that the AhR triggering by specific ligands during inflammatory conditions results in decreased IFNγ, IL-6, IL-12, TNF, IL-7, and IL-17, along with reduced microbial translocation and fibrosis in the gut, thus putational could control IBD (Pernomian et al., Clin Rev Allergy Immunol, 2020).

This experiment utilized a cell line of CaCo₂ stably expressing an AhR-response element. Cells were treated with indicated test articles for 24 hours (FICZ was used as a positive control). Results of the experiment are summarized in FIG. 14, showing that PA-F4 dose dependently upregulated AhR activity for up to 7-fold compared with the control group at 200 ug/mL. This suggests that ON101 and PAF4 may attenuate autoimmune disease by up-regulating the AhR signaling pathway.

Taken together, this experiment illustrates that PA-F4 and ON101 dose dependently up-regulate the AhR signaling pathway, providing insight into treatment options for autoimmune diseases such as such as psoriasis and inflammatory bowel disease (IBD).

(vi) PA-F4 Suppresses Dendritic Cell Activation and Maturation

Dendritic cells (DCs) are potent and versatile antigen-presenting cells, and their ability to migrate is key for the initiation of protective pro-inflammatory as well as tolerogenic immune responses. The abnormal activation of DCs may cause autoimmune diseases and allergic symptoms including psoriasis and allergic contact dermatitis in skin (Worbs et al., Nat Rev Immunol, 2017).

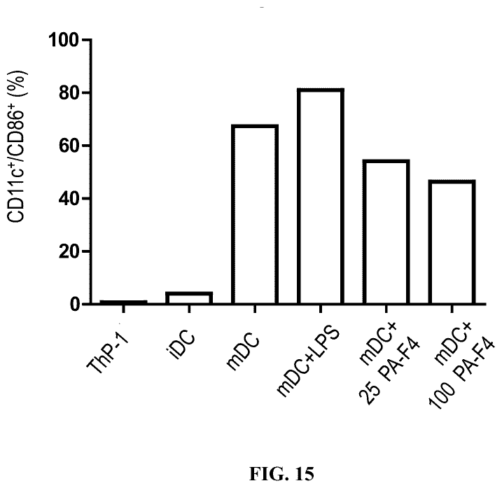

This experiment utilized a THP-1 polarized dendritic cell model. Results are shown in FIG. 15 and Table 5, demonstrating that PA-F4 treatment attenuated the maturation of DCs. This suggests that PA-F4 may improve DCs over-activated associated diseases.

TABLE 5
PA-F4 Suppressing Dendritic Cell Activation and Maturation

| | Procedure | Serum | GM-CSF | IL-4 | TNF-a | Ionomycin | Time |
|---|---|---|---|---|---|---|---|
| 1 | THP-1 | 10% FBS | — | — | — | — | — |
| | THP-1→iDC | 10% FBS | 100 ng/ml | 100 ng/ml | — | — | 4 day |
| | | | | | | | (Refresh media at day 2) |
| 2 | iDC→mDC | 10% FBS | 100 ng/ml | 200 ng/ml | 20 ng/ml | 200 ng/ml | Media removed, |
| | (fix cell density) | | | | | | refresh media for 2 days |

Taken together, this experiment illustrates that PA-F4 suppresses dendritic cell activation and maturation, providing insight into treatment options for various autoimmune diseases such as psoriasis and allergic contact dermatitis.

In sum, the experimental results obtained from this example show that the PA-F4 topical formulations investigated herein can modulate the development of autoimmune responses and diseases, such as alleviate psoriatic symptoms including erythema and plaque, suppress pathways involved in immune activation such as IL-17 and IL-23 pathways, up-regulate skin barrier-related gene expressions such as filaggrin, loricrin, homerin, and desmocollin and keratinocyte differentiation markers such as K1, up-regulate pathways involved in attenuating autoimmune responses such as AhR signaling, and suppress dendritic cell activation and maturation. Such results indicate that PA-F4 would benefit treatment of various autoimmune diseases, for example, psoriasis, atopic dermatitis (e.g., intrinsic atopic dermatitis or extrinsic atopic dermatitis), asthma, allergic rhinitis, food allergy, hay fever, inflammatory bowel disease (IBD), and allergic contact dermatitis.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for inhibiting immune responses, comprising administering to a subject in need thereof an effective amount of a composition comprising salvigenin and a carrier, optionally wherein the composition is a pharmaceutical composition, which further comprises a pharmaceutically acceptable carrier.

2. The method of claim 1, wherein the composition further comprises cirsimaritin, rosmarinic acid, carvacrol, or a combination thereof.

3. The method of claim 1, wherein the composition comprises a Plectranthus amboinicus (PA) extract.

4. The method of claim 3, wherein the PA extract is prepared by a process comprising:
(i) mixing a part of PA with an extracting solution to produce a first PA extract;
(ii) filtrating and concentrating the first PA extract to produce a concentrated PA extract;
(iii) contacting the concentrated PA extract onto a hydrophobic interaction chromatography resin; and
(iv) eluting the column with a eluent solution to product the PA extract.

5. The method of claim 4, wherein the part of PA in step (i) is an above-ground part.

6. The method of claim 4, wherein the extracting solution comprises a solvent having a polarity index of about 2.9 to 6.6; optionally wherein the extracting solution is acetone, butyl methyl ether, ethanol, ethyl acetate, isopropyl alcohol, methanol, or a mixture thereof.

7. The method of claim 4, wherein the eluent solution comprises a solvent having a polarity index of about 2.1-5.4; optionally wherein the eluent solution comprises a mixture of at least two solvents selected from the group consisting of acetone, ethanol, ethyl acetate, and hexane.

8. The method of claim 1, wherein the composition further comprises asiaticoside.

9. The method of claim 8, wherein the composition further comprises a Centella asiatica (CA) extract, which comprises the asiaticoside.

10. The method of claim 1, wherein the subject is a human patient having or suspected of having an immune disorder, which optionally is an autoimmune disease or an allergic disorder.

11. The method of claim 10, wherein the human patient has an autoimmune disease, which is psoriasis, or inflammatory bowel disease (IBD); optionally wherein the IBD is Crohn's disease or ulcerative colitis.

12. The method of claim 10, wherein the human patient has an allergic disorder, which is atopic dermatitis, asthma, allergic rhinitis, food allergy, or hay fever.

13. The method of claim 12, wherein the human patient has atopic dermatitis, which optionally is intrinsic atopic dermatitis or extrinsic atopic dermatitis.

14. The method of claim 1, wherein the subject is a human patient having psoriasis or atopic dermatitis, which optionally is intrinsic atopic dermatitis or extrinsic dermatitis.
